(19) United States
(12) Patent Application Publication
(10) Pub. No.: US 2012/0190119 A1
(43) Pub. Date: Jul. 26, 2012

(54) METHOD AND SYSTEM FOR MODULATING SUBSTANCES IN THE FORMATION VITAMINS

(76) Inventor: WILLIAM J. McFAUL, JACKSON, NJ (US)

(21) Appl. No.: 13/013,216

(22) Filed: Jan. 25, 2011

(51) Int. Cl. G01N 33/48

(52) U.S. Cl. 436/74; 436/90

(57) ABSTRACT

A method for establishing a treatment for promoting the formation of Vitamins in an individual. The measured or assessed level of substances in the body are compared in order to determine the treatment to provide to an individual.

### BACKGROUND OF THE INVENTION

1. Field of the Invention

The present invention relates to the field of modulated pairs. In particular the present invention is directed to the method and system for modulating substances in the formation of vitamins in the body.

2. Description of the Related Technology

All forms of life are supported by a variety of substances; including but not limited to hormones, proteins, peptides, amino acids, minerals (ions), vitamins (chemicals or compounds that function as vitamins) and bacteria. These substances create the homeostasis essential to maintain life for humans as well as plants and animals.

Pairs of substances affect levels of each other in the body. Additionally, certain other substances modulate (regulate) levels of the pairs of substances. Furthermore, certain substances are a part of more than one modulated pair of substances. These relationships can be utilized in order to determine various health parameters of an individual, however, to date; these corollary relationships have not been established in order to effectively use them.

Therefore, there is a need in the field to identify and utilize the homeostatic relationship between the substances that are important to the body's functioning via the measuring of various levels of the substances that are part of these pairings. There is also a need to utilize the homeostatic relationships in order to develop methods and systems for promoting the formation of essential components important for a healthy individual.

### SUMMARY OF THE INVENTION

An object of the invention is a method of establishing a treatment based upon the relationship of substances for forming vitamins.

Another object of the invention is a system for establishing balance in promoting the formation of vitamins.

An aspect of the present invention may be a method for establishing a treatment for promoting the formation of Vitamin C in an individual: measuring a level of calcium in the individual; measuring a level of cysteine in the individual; measuring a level of magnesium in the individual; comparing the measured levels; and establishing a treatment based upon the measured levels.

Another aspect of the present invention may be a method for establishing a treatment for promoting the formation of Vitamin D in an individual: measuring a level of phosphorous in the individual; measuring a level of tyrosine in the individual; measuring a level of calcium in the individual; comparing the measured levels; and establishing a treatment based upon the measured levels.

Still yet another aspect of the invention may be a method for establishing a treatment for promoting the formation of Vitamin B9 in an individual: measuring a level of phosphorus in the individual; measuring a level of cysteine in the individual; measuring a level of sulfur in the individual; comparing the measured levels; and establishing a treatment based upon the measured levels.

These and various other advantages and features of novelty that characterize the invention are pointed out with particularity in the claims annexed hereto and forming a part hereof. However, for a better understanding of the invention, its advantages, and the objects obtained by its use, reference should be made to the drawings which form a further part hereof, and to the accompanying descriptive matter, in which there is illustrated and described a preferred embodiment of the invention.

### BRIEF DESCRIPTION OF THE DRAWINGS

FIG. 1 illustrates the relationship of a modulated pair.

FIG. 2 shows the relationship between calcium, cysteine and magnesium.

FIG. 3 is a flow chart of the method for establishing a treatment based upon the relationship of the substances shown in FIG. 2.

FIG. 4 is a schematic diagram showing the system for measuring the levels of substances contained within the body.

FIG. 5 shows the method of using the system for measuring the levels of substances within an individual, in accordance with an embodiment of the present invention.

FIG. 6 shows the relationship between phosphorous, tyrosine and calcium.

FIG. 7 is a flow chart of the method for establishing a treatment based upon the relationship of the substances shown in FIG. 6.

FIG. 8 shows the relationship between phosphorous, cysteine and sulfur.

FIG. 9 is a flow chart of the method for establishing a treatment based upon the relationship of the substances shown in FIG. 8.

FIG. 10 shows the relationship between copper, glutamic acid, and zinc.

FIG. 11 is a flow chart of the method for establishing a treatment based upon the relationship of the substances shown in FIG. 10.

FIG. 12 shows the relationship between BDNF, NT-3 and NNT-1.

FIG. 13 is a flow chart of the method for establishing a treatment based upon the relationship of the substances shown in FIG. 12.

FIG. 14 shows the relationship between BDNF, P75 neurotrophic and receptor.

FIG. 15 is a flow chart of the method for establishing a treatment based upon the relationship of the substances shown in FIG. 14.

FIG. 16 shows the relationship between COX-1, COX-3 and COX-2.

FIG. 17 is a flow chart of the method for establishing a treatment based upon the relationship of the substances shown in FIG. 16.

FIG. 18 shows the relationship between CPS1, CPS2 and SIRT 5.

FIG. 19 is a flow chart of the method for establishing a treatment based upon the relationship of the substances shown in FIG. 18.

FIG. 20 shows the relationship between sodium, lithium and chloride.

FIG. 21 is a flow chart of the method for establishing a treatment based upon the relationship of the substances shown in FIG. 20.

FIG. 22 shows the relationship between calcium, lysine and magnesium.

FIG. 23 is a flow chart of the method for establishing a treatment based upon the relationship of the substances shown in FIG. 22.

FIG. 24 shows the relationship between troponin C, troponin I and troponin T.

FIG. 25 is a flow chart of the method for establishing a treatment based upon the relationship of the substances shown in FIG. 24.

FIG. 26 shows the relationship between TNNT-1, TNNT-3 and TNNT-2.

FIG. 27 is a flow chart of the method for establishing a treatment based upon the relationship of the substances shown in FIG. 26.

FIG. 28 shows the relationship between TNNI-1, TNNI-3 and TNNI-2.

FIG. 29 is a flow chart of the method for establishing a treatment based upon the relationship of the substances shown in FIG. 28.

FIG. 30 shows the relationship between EGFR-4, EGFR-1 and GRB7.

FIG. 31 is a flow chart of the method for establishing a treatment based upon the relationship of the substances shown in FIG. 30.

FIG. 32 shows the relationship between EGFR-2, EGFR-1 and EGFR-3.

FIG. 33 is a flow chart of the method for establishing a treatment based upon the relationship of the substances shown in FIG. 32.

FIG. 34 shows the relationship between MBD2, MBD1 and MeCP2.

FIG. 35 is a flow chart of the method for establishing a treatment based upon the relationship of the substances shown in FIG. 34.

FIG. 36 shows the relationship between MBD3, MBD1 and MBD4.

FIG. 37 is a flow chart of the method for establishing a treatment based upon the relationship of the substances shown in FIG. 36.

FIG. 38 shows the relationship between Adrenaline, Oxytocin and Cortisol.

FIG. 39 is a flow chart of the method for establishing a treatment based upon the relationship of the substances shown in FIG. 38.

FIG. 40 shows the relationship between sulfur, histidine and iron.

FIG. 41 is a flow chart of the method for establishing a treatment based upon the relationship of the substances shown in FIG. 40.

FIG. 42 shows the relationship between E3, E1 and pregnenolone.

FIG. 43 is a flow chart of the method for establishing a treatment based upon the relationship of the substances shown in FIG. 42.

FIG. 44 shows the relationship between testosterone, E1 and E2.

FIG. 45 is a flow chart of the method for establishing a treatment based upon the relationship of the substances shown in FIG. 44.

FIG. 46 shows the relationship between phosphorous, tyrosine and manganese.

FIG. 47 is a flow chart of the method for establishing a treatment based upon the relationship of the substances shown in FIG. 46.

### DETAILED DESCRIPTION OF THE PREFERRED EMBODIMENT(S)

Modulated pairs of substances create the homeostasis essential to maintain life for humans as well as animals.

Several terms are used herein and are defined as follows:

The term “modulator” means an agent, substance or mechanism that facilitates the maintenance of homeostasis between pairs. “Pairs” may be hormones, minerals, proteins, amino acids, bacteria, virus, gases and natural processes. When a pair is “modulated” the modulator can decrease intensity of stimulatory processes or substances, or increase intensity of compensatory ones.

The term “catalyst” may mean the driver, which initiates modulation to take place. Such catalysts can include enzymes, hormones, RNA, gases and countless signaling mechanisms.

The term “Homeostatic Relationship (Homeostasis)” means the balance or equilibrium between two substances. The relationship does not have to be 50%-50% but can vary depending upon the two substances in question.

The term “Extracellular Matter (ECM)” means all body fluid outside of cells and blood; including but not limited to cerebrospinal fluid, lymph, chyle, interstitial fluid, saliva, transcellular fluid, amniotic fluid, sperm, and sputum.

The term “Mental Functioning Activity (MFA)” means the rate of activity attributable to the neural firing rate in the brain and elsewhere in the body.

The invention addresses the diagnostic analysis of the corollary relationships between substances that provide and maintain homeostasis in an individual and promote the formation of vitamins.

The data for measuring homeostasis between substances within a modulated pair can vary from one individual or specimen to another due to environmental factors, basic metabolism, genetic make-up and other factors. Data derived from the application of the invention will allow for diagnostic analysis and control of levels of the substances in order to prevent their depletion, to maintain adequate levels to prevent disruption as well as to create recommendations for which substances may counteract with others and the levels of which substances must be modified on an ongoing basis in order to provide optimal health/well being through homeostasis.

Since nutrients, such as vitamins, minerals, electrolytes, enzymes, amino acids, proteins, etc., are essential to create defenses for the organs, glands and operating systems of the body, as well as to provide the energy to sustain life, establishing corollary relationships between substances that may require “nutrients” for their production will enable cross-category analysis to prevent depletion of the body's defense in order to prevent illness as well as to increase levels of nutritional energy to strengthen the body to offset the effects of existing ailments and diseases.

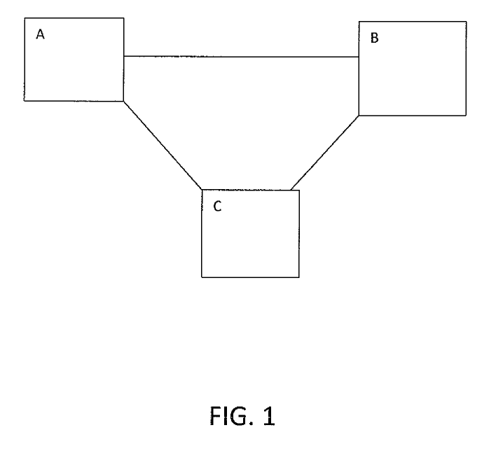

The concept of modulation in relation to homeostatic pairs is illustrated in FIG. 1. This example illustrates a relationship between two substances, A and B, and the existence of a third substance C that serves to support A or B in order to attempt to maintain an adequate level of homeostasis (balance) between the two substances.

Some examples of modulated pairs that operate within an individual that can affect vitamin formation may be as follow: calcium and cysteine modulated by magnesium having a role in the process for the formation of Vitamin C, this may also play a role in the formation of the constituents of calnexin; phosphorous and tyrosine modulated by calcium having a role in the formation of Vitamin D; phosphorous and cysteine modulated by sulfur having a role in the formation of Vitamin B9 (Folic Acid); copper and glutamic acid modulated by zinc having a role in the formation of Vitamin B6 (pyridoxal phosphate).

Other examples of modulated pairs may be as follows: brain-derived neurotrophic factor (BDNF) and Neurotropin-3 (NT-3) modulated by novel-neurotrophin-1 (NNT-1) having a role in neural cell proliferation; Brain-derived neurotrophic factor (BDNF) and P75 neurotrophic recepter modulated by nerve growth factor (NGF) having a role in the formation of neuron synthesis; cyclooxygenase-1 (COX-1) and Cyclooxygenase-3 (COX-3) modulated by Cyclooxygenase-2 (COX-2) playing a role in formation of enzymes for lipid conversions for smooth muscle tissue; Carbamoyl phosphate synthase 1 (CPS1) and Carbamoyl Phosphate Synthase 2 (CPS2) modulated by SIRT 5 having a role in the conversion of pyrimidines; sodium and lithium modulated by chloride having a role in the formation of a signaling molecule for oxytocin to cortisol; calcium and cysteine modulated by magnesium having a role in the formation of consitutents of calnexin; calcium and lysine modulated by magnesium having a role in the formation of consituents of calreticulin; troponin C and troponin I modulated by troponin T having a role in the formation of constituents of troponin necessary for homeostasis of muscle contraction; troponin T slow subunit 1 (TNNT-1) and troponin fast subunit 3 (TNNT-3) modulated by troponin cardiac (TNNT-2) having a role in the formation of troponin T; troponin 1 slow subunit (TNNI-1) and troponin fast subunit (TNNI-3) modulated by troponin cardiac (TNNI-2) having a role in the formation of troponin I; Epidermal Growth Factor Receptor 4 (EGFR4) and Epidermal Growth Factor Receptor 1 (EGFR1) modulated by Growth Factor Receptor-bond Protein 7 (GRB7) having a role in the formation of factors for cell proliferation and adhesion; Epidermal Growth Factor Receptor 2 (EGFR2) and Epidermal Growth Factor Receptor 1 (EGFR1) modulated by Epidermal Growth Factor Receptor 3 (EGFR3) having the role/process in/for the formation of factors for cell proliferation and adhesion; Methyl-CpG-Binding Domain Protein 2(MBD2) and Methyl-CpG Binding Domain Protein 1(MBD1) modulated by Methyl-CPG-Binding Domain Protein 2 Variant (MeCP2) having the a role in the formation of constituents of chromatin; Methyl-CpG-Binding Domain Protein 3 (MBD3) and Methyl-CpG-Binding Domain Protein 1 (MBD1) modulated by Methyl-CpG-Binding Domain Protein 4 (MBD4) having the role/process for the formation of protein constituents for maintenance of chromatin; adrenaline and oxytocin modulated by cortisol having a role in regulating anger and contentment; Sulfur—histidine modulated by iron playing a role in the process for the formation of Histidine-rich Glycoprotein (HRG); Estradiol (E3) and Estrone (E1) modulated by pregnenolone having a role in the process for the formation of Estrogen; Testosterone and Estrone (E1) modulated by Estriol (E2) having a role in the process for the regulation of Estrogen in males; and phosphorous and tyrosine modulated by manganese having the role a signaling of the Abl protein.

It should be noted that some substances can function as part of a pair and also be a modulator for another pair. Additionally, it should be understood that while specific processes and roles are noted above as being affected by the pairs, other processes and roles may also be affected.

Correlation between base values expressed when the range of homeostasis is determined and individual test results can be used as a means of assessing levels or disruptions between pairs or the existence of increases in the levels of modulators in order to maintain homeostasis. Methods of diagnostic analysis may also detect the presence of catalysts as markers for the existence of a disruption within a modulated pair that the body is in the process of correcting. Detection of such catalysts may be included as a factor in the process of analyzing the correlation of substances within modulated pairs.

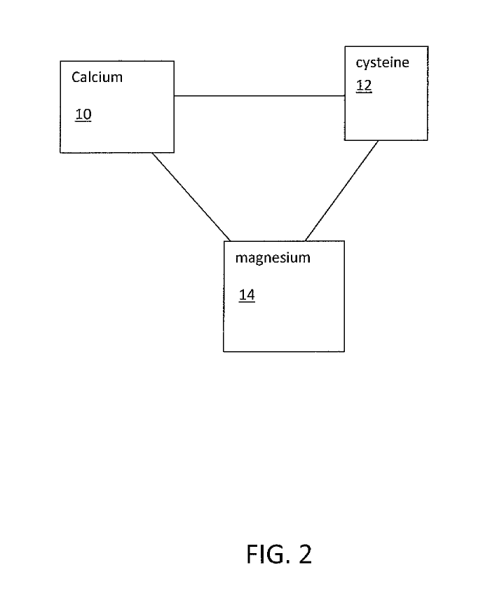

Described herein is an example of the determination of homeostatic levels of the sets of modulated pairs discussed above. The first set is the pair of calcium 10 and cysteine 12, which is modulated by magnesium 14. This relationship is shown in FIG. 2. This relationship plays a role in the formation of Vitamin C. This relationship may also play a role in the formation of the constituents of calnexin.

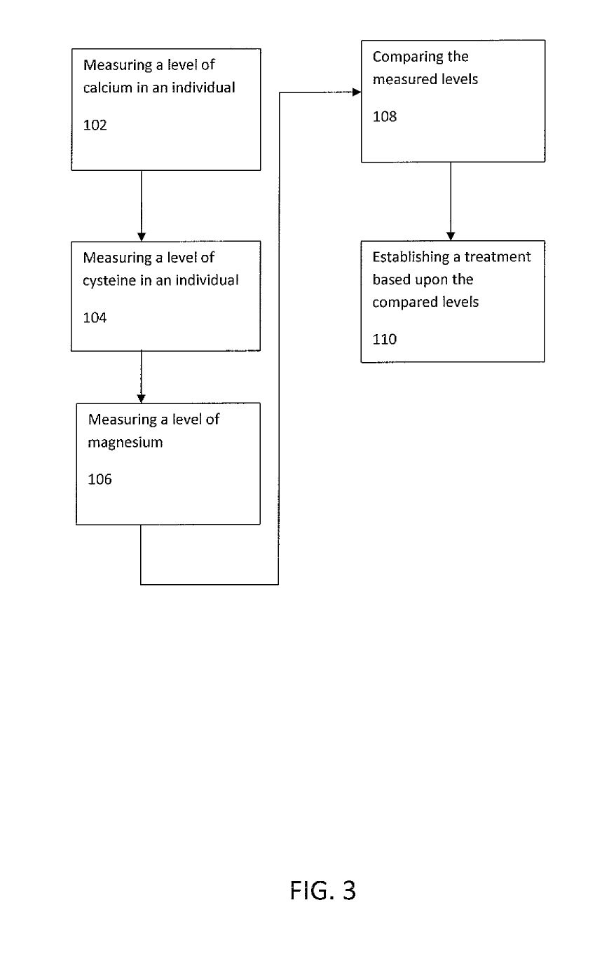

FIG. 3 shows the method of evaluating the homeostatic levels of one set of the modulated pairs in order to provide a base index for administering medication and performing analysis. In step 102, the level of calcium 10 is measured in an individual. The level of calcium 10 may be measured by a variety of diagnostic tests that include but are not limited to the analysis of diagnostic imaging that indicates activity and the analysis of various fluids and excretions within and produced by the body. Examples of these fluids and excretions include: blood and its components; serum; urine; fecal matter; collagen; chyle; interstitial fluid (tissue fluid); lymph; extracellular fluid; amniotic fluid; sweat tears; saliva; mucus; phlegm; hair; fingernails; bone marrow.

In addition to the conventional diagnostic analysis of levels of substances from the aforementioned sources, various existing and yet to be developed diagnostic processes that measure outcomes resulting from these substances can be utilized. These devices may include EEG, PET scans, use of MEG machines, SPECT analysis, functional and diffusion MRI technologies as well as other iterations thereof (fMRI and dMRI respectively), CT scans, and ultrasound. Correlation between base values expressed when the range of homeostasis is determined and individual test results may then be used as a means of assessing levels or disruptions between pairs or the existence of increases in the levels of modulators in order to maintain homeostasis. Methods of diagnostic analysis may also detect the presence of catalysts as markers for the existence of a disruption within a modulated pair that the body is in the process of remediating. Detection of such catalysts is included as a factor in the process of analyzing the correlation of substances within modulated pairs.

This invention provides the biological foundation that will enable monitoring relationships within modulated pairs of substances as they relate to medical care; including wellness, prevention and treatment pertaining to the mind (brain) and body.

The measured level of calcium 10 in the body or environment may be expressed in various units depending upon the test employed in order to measure the level of the calcium 10. This level can be expressed by the variable X. There is a preferred range in which X may fall that varies depending upon the type of test that is used in order to measure the level of calcium 10. In an embodiment of the present invention a standard type of test is used.

In step 104, the level of cysteine 12 in an individual is measured. This level can be measured using one of the various processes listed above. The measured level of cysteine 12 is preferably expressed in the same units of measurement as that used in the measurement of calcium 10 so as to more readily compare the levels with respect to each other in order to obtain a base level. This level can be expressed by the variable Y. There is a preferred range in which Y may fall that varies depending upon the type of test that is used in order to measure the level of cysteine 12. In an embodiment of the present invention a standard type of test is used, such as a blood test.

In step 106, the level of magnesium 14 in an individual is measured. This level can be measured using one of the various processes listed above. The measured level of magnesium 14 is preferably expressed in the same units of measurement as that used in the measurement of calcium 10 so as to more readily compare the levels with respect to each other in order to obtain a base level. This level can be expressed by the variable Z. There is a preferred range in which Z may fall that varies depending upon the type of test that is used in order to measure the level of magnesium 14. In an embodiment of the present invention a standard type of test is used.

In step 108, the measured levels X, Y and Z are then compared to standard levels. This may be done in a number of different ways. One possible way in which this can be accomplished is to total the values of all of the levels (X+Y+Z) in order to establish sum level C. It is also possible to establish acceptable ratios for the values X:Y, X:Z and Y:Z. The sum level C can be used in step 110 in order to establish a treatment or diagnosis of the individual that can be used in determining the provision of medication and other matters related to the measured levels. In particular the levels may be used to determine if more magnesium 14 may need to be supplied in order to place the level of calcium 10 or cysteine 12 back to proper levels and to promote the formation of Vitamin C.

An example of this is provided below using hypothetical numbers so as to make understanding of the process easier.

Measuring calcium 10 via a blood test results in a number for X that results in a number 6 in the appropriate units, from within a range of 1-10. Measuring cysteine 12 via a blood test results in a number for Y of 5, within the appropriate units, from within a range of 1-10. Measuring magnesium 14 via a blood test results in a number for Z of 6, within the appropriate units, from within a range of between 1-10. These numbers are totaled and result in a C value of 17. A value for C between 10 and 20 may indicate a normal level.

A value for C that falls below 10 or above 20 may signal the need to provide a treatment that administers additional magnesium 14 to the individual. The magnesium 14 may be administered via pill form or some other acceptable means. Monitoring of the individual may be performed periodically until the levels return to accepted ranges. Alternatively, one of the substances may be removed from an individual's diet. It is also contemplated that one of the other substances, such as calcium 10 or cysteine 12 may be added or removed.

The assessment of the modulated substances enables the provision of consulting/counseling services relative to the biological foundation for and between the substances associated with the formation of Vitamin C.

The usage of these methods may also be used with devices that will measure levels of the substances contained within the body and listed above. The devices may be used to assess the corollary relationships between the substances in order to compare them to standard biodiagnostic baseline assessment.

The usage of these methods can also be used for assessment of homeostatic imbalances of relationships within modulated pairs that can enable therapies to correct these disruptions instead of waiting to the point where mental or physical consequences will require expensive or sophisticated treatment of ailments or diseases that are the outcomes.

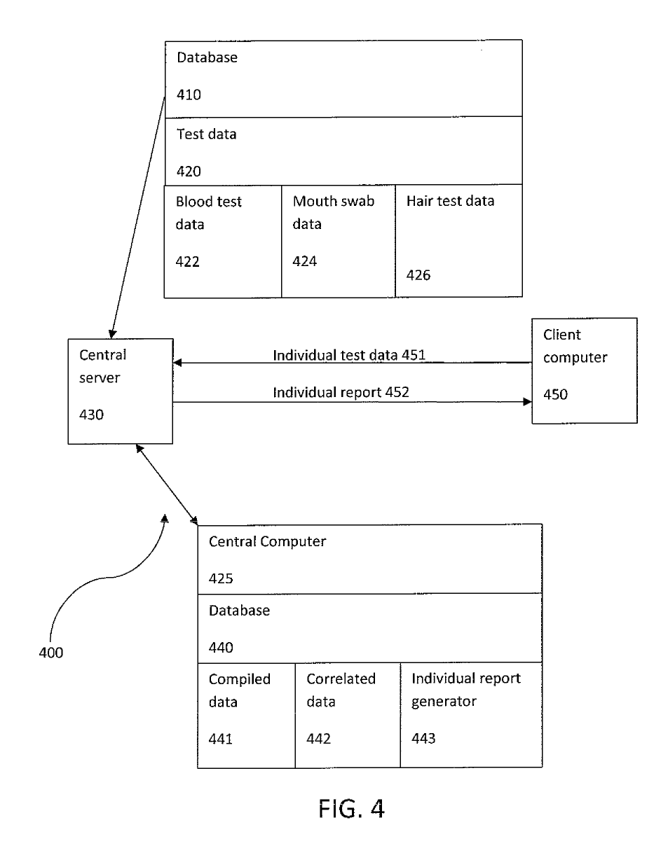

The usage of these methods can also be used for the design of devices to correlate the relationship between the various substances contained in the body as listed in this invention. These devices can include but should not be limited to ones used within the veins and arteries, ongoing assessment as implanted devices, monitoring devices for home use or those used to perform diagnostic assessments in physicians offices, hospitals, diagnostic laboratories, home care or any other setting where the analysis of these substances contained in the blood are measured and compared to each other to perform assessments to maintain proper health and well being. A schematic of the system diagram is shown in FIG. 4.

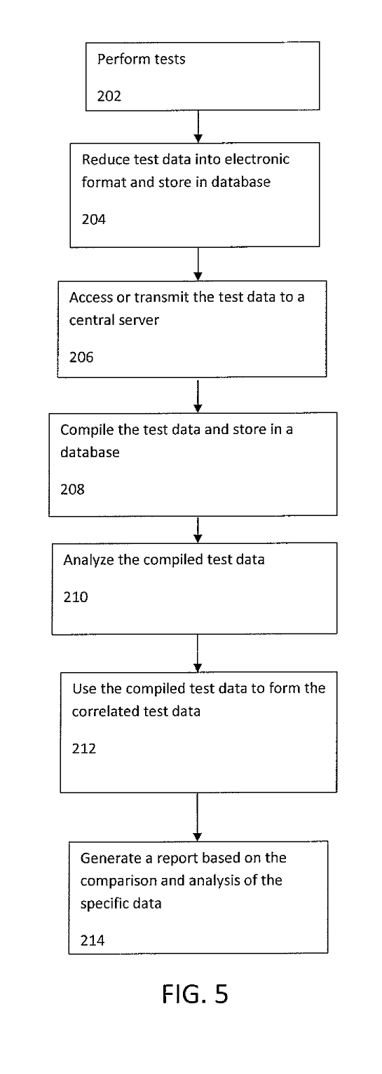

FIG. 4 shows a personalized report generator system 400 designed and arranged in accordance with an embodiment of the present invention. It should be understood that system 400 shows the basic components of the system and that more or less individualized components may be provided. FIG. 5 is a flow chart showing the steps involved in using the system 400.

The personalized report generator system 400 comprises a database 410 in which may be stored various test results. The data is stored in the database 410 as test data 420. The test data 420 is in a format that is capable of being manipulated by computers, processors, etc. The test data 420 may comprise various substance levels. In an exemplary embodiment of the present invention and shown in step 202, tests are performed on individual patients. In step 204, the results of these tests are then reduced into an electronic format in order to produce the test data 420. For example, the database 410 may store blood test data 422, mouth swab data 424 and hair test data 426. Other tests may include urine; fecal matter; collagen; chyle; interstitial fluid (tissue fluid); lymph; extracellular fluid; amniotic fluid; sweat tears; saliva; mucus; phlegm; hair; fingernails; and bone marrow. Each of these various tests may screen for a plurality of substances. As an example, the blood test data 422 may screen for calcium 10, cysteine 12 and magnesium 14.

The test data 420 may be organized and stored at the database 410. Alternatively, satellite locations may each maintain their own databases 410 that control their own records. In step 206, these locations may be accessed periodically by the central server 430 or the various databases 410 may transmit their results to a central location that is then accessible by a central server 430. Preferably the test data 420 is only accessed or transmitted when a certain threshold number of results are obtained. For example the test data 420 may not be sent to the central server 430 until at least one hundred tests are prepared to be transmitted.

In step 208, the transmitted test data 420 is compiled and stored as compiled test data 441. The central server 430 may keep the sent or accessed compiled test data 441 stored at its location in the central database 440, or the system 400 may provide a distributed storage system that stores the compiled test data 441 at external locations as well as the central database 440. However it should be understood that when compiled test data 441 is stored at distributed sites the system still functions as if they were stored at the central database 440. The compiled test data 441 may be stored so that the origin of the data is maintained in an anonymous fashion. In some instances tracking of the origin of a test result may only occur when a personalized report is requested.

In step 210, the compiled test data 441 is then analyzed in order to determine significant figures for conducting analysis. The analysis may be accomplished with the usage of a computer(s) 425 of which the server 430 may be a part of, as well as the database 410. The analysis may be accomplished by statistically compiling and correlating the results for one specific test or by compiling the data for two separate items that are believed to have a relationship. Specific values from the plurality of compiled test data 441 having a similar feature may be analyzed. This may be accomplished through the application of known statistical algorithms. In step 212, the statistically compiled test data 441 may be used to form the correlated data 442. In step 214, the statistically correlated data 442 are then used by the individual report generator 443 to form the personalized medical prescription and diagnosis. Upon receipt of individual test data 451 from a client computer 450, the individual report generator 443 processes the data and transmits an individual report 452 to a client computer 450.

The compiled test data 441 may be periodically updated from the databases 410 and through the addition of more collected test data. The updating of the compiled test data 441 may provide additional information with which to refine the correlated data 442 and to refine the ranges used by the individual report generator 443 when producing the personalized medical prescription and diagnosis. Additionally the updating of the compiled test data 441 may be a continuous feature wherein the addition of new information is immediately reflected in the correlated data 442. This may provide for a constantly fluid analysis of compiled test data 441 that may reflect changes in population and diets in society.

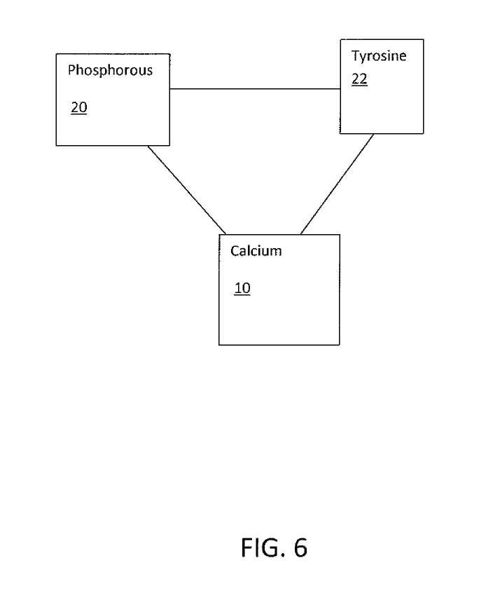

Described herein is another example of the determination of homeostatic levels of the sets of modulated pairs discussed above. The pair of phosphorous 20 and tyrosine 22 is modulated by calcium 10 and plays a role in the formation of Vitamin D. This relationship is shown in FIG. 6.

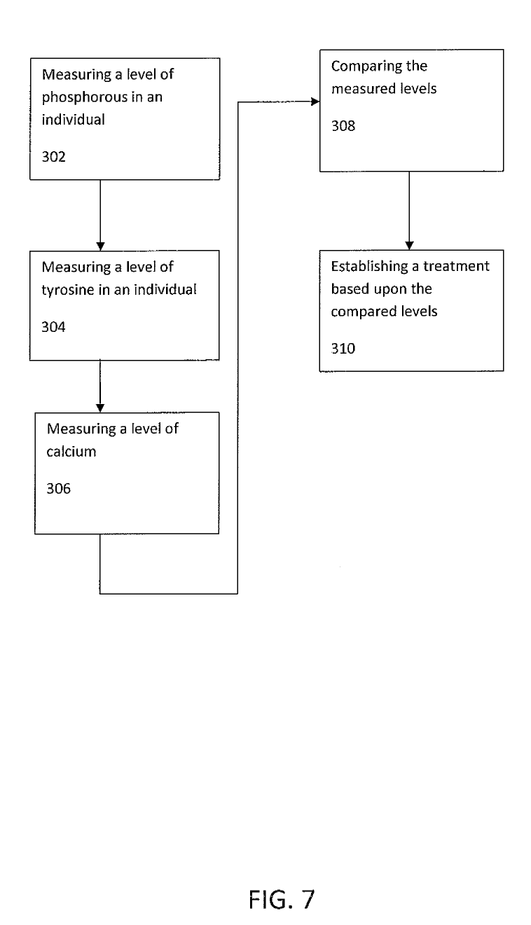

FIG. 7 shows the method of evaluating the homeostatic levels the modulated pairs in order to provide a base index for administering medication and performing analysis. In step 302, the level of phosphorous 20 is measured in an individual. The level of phosphorous 20 as well the other substances discussed within this application may be determined in the same manner as discussed above with respect to the testing methods set forth in FIG. 3.

The measured level of phosphorous 20 in the body or environment may be expressed in various units depending upon the test employed in order to measure the level of the phosphorous 20. This level can be expressed by the variable X. There is a preferred range in which X may fall that varies depending upon the type of test that is used in order to measure the level of phosphorous 20. In an embodiment of the present invention a standard type of test is used.

In step 304, the level of tyrosine 22 in an individual is measured. This level can be measured using one of the various processes listed above. The measured level of tyrosine 22 is preferably expressed in the same units of measurement as that used in the measurement of phosphorous 20 so as to more readily compare the levels with respect to each other in order to obtain a base level. This level can be expressed by the variable Y. There is a preferred range in which Y may fall that varies depending upon the type of test that is used in order to measure the level of tyrosine 22. In an embodiment of the present invention a standard type of test is used, such as a blood test.

In step 306, the level of calcium 10 in an individual is measured. This level can be measured using one of the various processes listed above. The measured level of calcium 10 is preferably expressed in the same units of measurement as that used in the measurement of phosphorous 20 so as to more readily compare the levels with respect to each other in order to obtain a base level. This level can be expressed by the variable Z. There is a preferred range in which Z may fall that varies depending upon the type of test that is used in order to measure the level of calcium 10. In an embodiment of the present invention a standard type of test is used.

In step 308, the measured levels X, Y and Z are then compared to standard levels. This may be done in a number of different ways. One possible way in which this can be accomplished is to total the values of all of the levels (X+Y+Z) in order to establish sum level C. This sum level C can be used in step 310 in order to establish a diagnosis of the individual that can be used in determining the provision of medication and other matters related to the measured levels. In particular the levels may be used to determine if more calcium 10 may need to be supplied in order to place the level of phosphorous 20 or tyrosine 22 back to proper levels and to further promote the formation of Vitamin D. A value for C that falls below or above the predetermined level may signal the need to administer additional calcium 10 to the individual. The calcium 10 may be administered via pill form or some other acceptable means. Alternatively, it may be suggested that an individual's behavior be modified in order to reduce levels of one of the substances. One of the substances may be removed from an individual's diet. It is also contemplated that one of the other substances, such as calcium 10 or phosphorus 20 may be added or removed.

The assessment of the modulated substances enables the provision of consulting/counseling services relative to the biological foundation for and between the substances associated with the formation of Vitamin D.

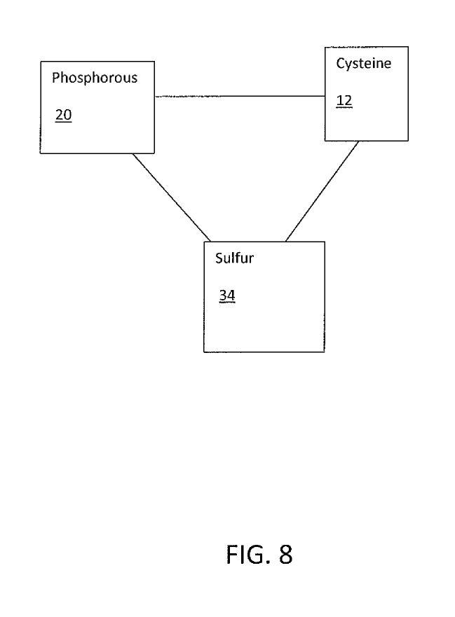

Described herein is another example of the determination of homeostatic levels of the sets of modulated pairs discussed above. The pair of phosphorous 20 and cysteine 12 is modulated by sulfur 34 and plays a role in the formation of Vitamin B9. This relationship is shown in FIG. 8.

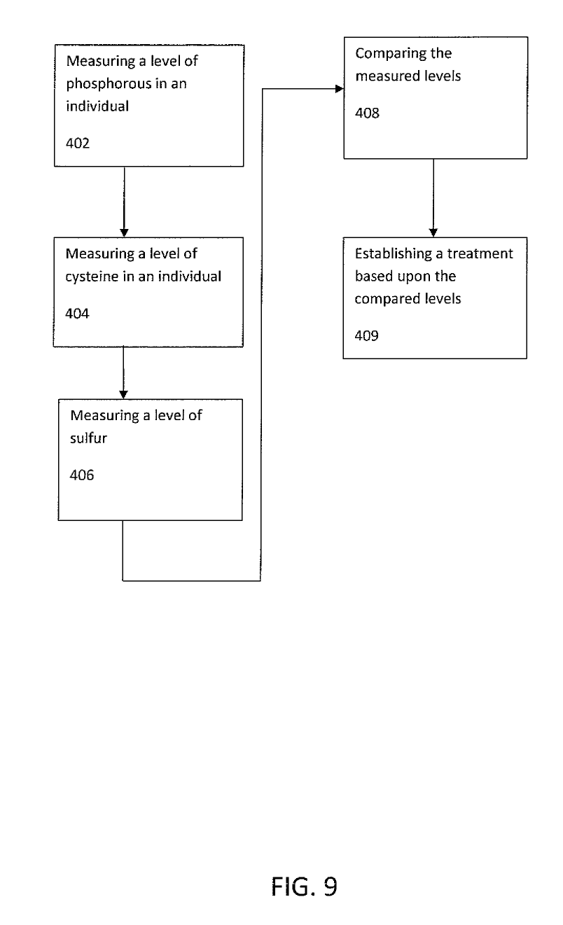

FIG. 9 shows the method of evaluating the homeostatic levels the modulated pairs in order to provide a base index for administering medication and performing analysis. In step 402, the level of phosphorous 20 is measured in an individual. The level of phosphorous 20 may be measured in the same manner as the substances discussed above.

The measured level of phosphorous 20 in the body or environment may be expressed in various units depending upon the test employed in order to measure the level of the phosphorous 20. This level can be expressed by the variable X. There is a preferred range in which X may fall that varies depending upon the type of test that is used in order to measure the level of phosphorous 20. In an embodiment of the present invention a standard type of test is used.

In step 404, the level of cysteine 12 in an individual is measured. This level can be measured using one of the various processes listed above. The measured level of cysteine 12 is preferably expressed in the same units of measurement as that used in the measurement of phosphorous 20 so as to more readily compare the levels with respect to each other in order to obtain a base level. This level can be expressed by the variable Y. There is a preferred range in which Y may fall that varies depending upon the type of test that is used in order to measure the level of cysteine 12. In an embodiment of the present invention a standard type of test is used, such as a blood test.

In step 406, the level of sulfur 34 in an individual is measured. This level can be measured using one of the various processes listed above. The measured level of sulfur 34 is preferably expressed in the same units of measurement as that used in the measurement of phosphorous 20 so as to more readily compare the levels with respect to each other in order to obtain a base level. This level can be expressed by the variable Z. There is a preferred range in which Z may fall that varies depending upon the type of test that is used in order to measure the level of sulfur 34. In an embodiment of the present invention a standard type of test is used.

In step 408, the measured levels X, Y and Z are then compared to standard levels. This may be done in a number of different ways. One possible way in which this can be accomplished is to total the values of all of the levels (X+Y+Z) in order to establish sum level C. This sum level C can be used in step 409 in order to establish a diagnosis of the individual that can be used in determining the provision of medication and other matters related to the measured levels. In particular the levels may be used to determine if more sulfur 34 may need to be supplied in order to place the level of phosphorous 20 or cysteine 12 back to proper levels and to further promote the formation of Vitamin B9. A value for C that falls below or above the predetermined level may signal the need to administer additional sulfur 34 to the individual. The sulfur 34 may be administered via pill form or some other acceptable means. Alternatively, it may be suggested that an individual's behavior be modified in order to reduce levels of one of the substances. One of the substances may be removed from an individual's diet. It is also contemplated that one of the other substances, may be added or removed.

The assessment of the modulated substances enables the provision of consulting/counseling services relative to the biological foundation for and between the substances associated with the formation of Vitamin B9.

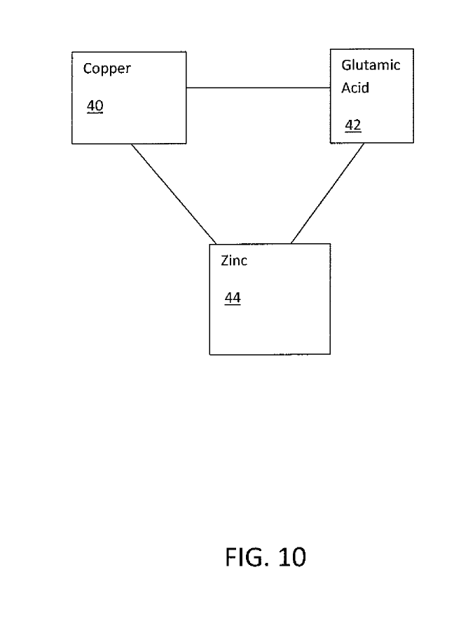

Described herein is another example of the determination of homeostatic levels of the sets of modulated pairs discussed above. The pair of copper 40 and glutamic acid 42 is modulated by zinc 44 and plays a role in the formation of Vitamin B6 (Pyridoxal phosphate). This relationship is shown in FIG. 10.

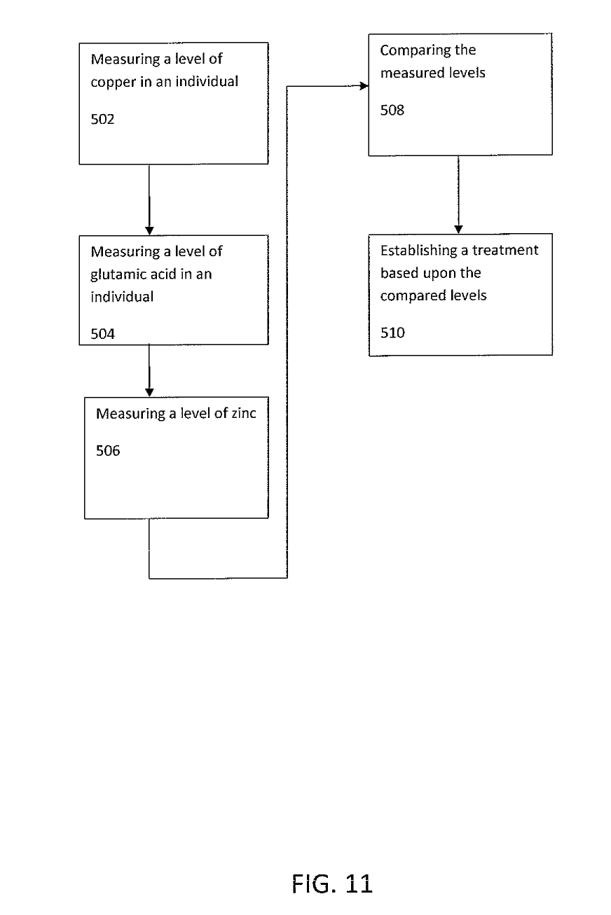

FIG. 11 shows the method of evaluating the homeostatic levels the modulated pairs in order to provide a base index for administering medication and performing analysis. In step 502, the level of copper 40 is measured in an individual. The level of copper 40 may be measured in the same manner as the substances discussed above.

The measured level of copper 40 in the body or environment may be expressed in various units depending upon the test employed in order to measure the level of the copper 40. This level can be expressed by the variable X. There is a preferred range in which X may fall that varies depending upon the type of test that is used in order to measure the level of copper 40. In an embodiment of the present invention a standard type of test is used.

In step 504, the level of glutamic acid 42 in an individual is measured. This level can be measured using one of the various processes listed above. The measured level of glutamic acid 42 is preferably expressed in the same units of measurement as that used in the measurement of copper 40 so as to more readily compare the levels with respect to each other in order to obtain a base level. This level can be expressed by the variable Y. There is a preferred range in which Y may fall that varies depending upon the type of test that is used in order to measure the level of glutamic acid 42. In an embodiment of the present invention a standard type of test is used, such as a blood test.

In step 506, the level of zinc 44 in an individual is measured. This level can be measured using one of the various processes listed above. The measured level of zinc 44 is preferably expressed in the same units of measurement as that used in the measurement of copper 40 so as to more readily compare the levels with respect to each other in order to obtain a base level. This level can be expressed by the variable Z. There is a preferred range in which Z may fall that varies depending upon the type of test that is used in order to measure the level of zinc 44. In an embodiment of the present invention a standard type of test is used.

In step 508, the measured levels X, Y and Z are then compared to standard levels. This may be done in a number of different ways. One possible way in which this can be accomplished is to total the values of all of the levels (X+Y+Z) in order to establish sum level C. This sum level C can be used in step 510 in order to establish a diagnosis of the individual that can be used in determining the provision of medication and other matters related to the measured levels. In particular the levels may be used to determine if more zinc 44 may need to be supplied in order to place the level of copper 40 or glutamic acid 42 back to proper levels and to further promote the formation of Vitamin B6. A value for C that falls below or above the predetermined level may signal the need to administer additional zinc 44 to the individual. The zinc 44 may be administered via pill form or some other acceptable means. Alternatively, it may be suggested that an individual's behavior be modified in order to reduce levels of one of the substances. One of the substances may be removed from an individual's diet. It is also contemplated that one of the other substances, may be added or removed.

The assessment of the modulated substances enables the provision of consulting/counseling services relative to the biological foundation for and between the substances associated with the formation of Vitamin B6.

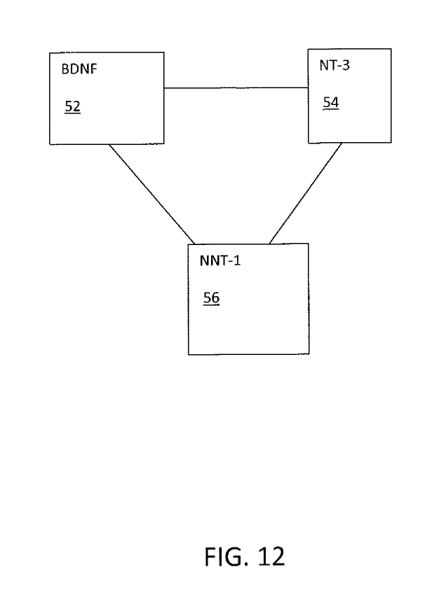

Another example of the determination of homeostatic levels of the sets of modulated pairs discussed above is now provided. The pair of BDNF 52 and NT-3 54 is modulated by NNT-1 56 and plays a role in neural cell proliferation. This relationship is shown in FIG. 12.

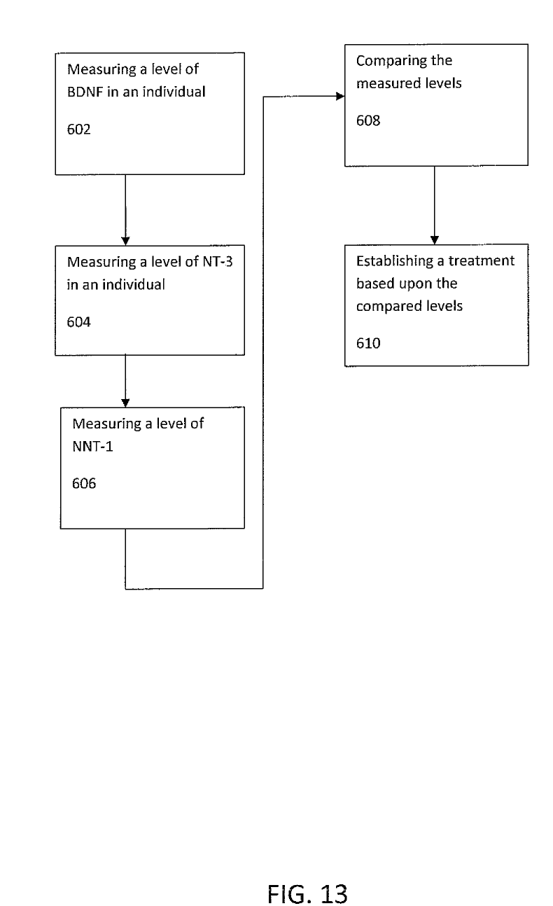

FIG. 13 shows the method of evaluating the homeostatic levels of the modulated pairs in order to provide a base index for administering medication and performing analysis. In step 602, the level of BDNF 52 is measured in an individual. The level of BDNF 52 may be measured in the same manner as the substances discussed above.

The measured level of BDNF 52 in the body or environment may be expressed in various units depending upon the test employed in order to measure the level of the BDNF 52. This level can be expressed by the variable X. There is a preferred range in which X may fall that varies depending upon the type of test that is used in order to measure the level of BDNF 52. In an embodiment of the present invention a standard type of test is used.

In step 604, the level of NT-3 54 in an individual is measured. This level can be measured using one of the various processes listed above. The measured level of NT-3 54 is preferably expressed in the same units of measurement as that used in the measurement of BDNF 52 so as to more readily compare the levels with respect to each other in order to obtain a base level. This level can be expressed by the variable Y. There is a preferred range in which Y may fall that varies depending upon the type of test that is used in order to measure the level of NT-3 54. In an embodiment of the present invention a standard type of test is used, such as a blood test.

In step 606, the level of NNT-1 56 in an individual is measured. This level can be measured using one of the various processes listed above. The measured level of NNT-1 56 is preferably expressed in the same units of measurement as that used in the measurement of BDNF 52 so as to more readily compare the levels with respect to each other in order to obtain a base level. This level can be expressed by the variable Z. There is a preferred range in which Z may fall that varies depending upon the type of test that is used in order to measure the level of NNT-1 56. In an embodiment of the present invention a standard type of test is used.

In step 608, the measured levels X, Y and Z are then compared to standard levels. This may be done in a number of different ways. One possible way in which this can be accomplished is to total the values of all of the levels (X+Y+Z) in order to establish sum level C. This sum level C can be used in step 610 in order to establish a diagnosis of the individual that can be used in determining the provision of medication and other matters related to the measured levels. In particular the levels may be used to determine if more NNT-1 56 may be needed to be supplied in order to place the level BDNF 52 or NT-3 54 back to proper levels and to further promote neural cell proliferation. A value for C that falls below or above the predetermined level may signal the need to administer additional NNT-1 56 to the individual. The NNT-1 56 may be administered via pill form or some other acceptable means. Alternatively, it may be suggested that an individual's behavior be modified in order to reduce levels of one of the substances.

The assessment of the modulated substances enables the provision of consulting/counseling services relative to the biological foundation for and between the substances associated with promoting neural cell proliferation.

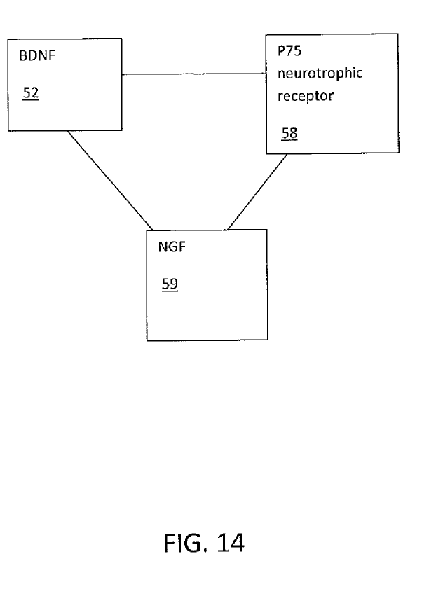

Another example of the determination of homeostatic levels of the sets of modulated pairs discussed above is now provided. The pair of BDNF 52 and P75 neurotrophic receptor 58 is modulated by NGF 59 and plays a role in neuron synthesis. This relationship is shown in FIG. 14.

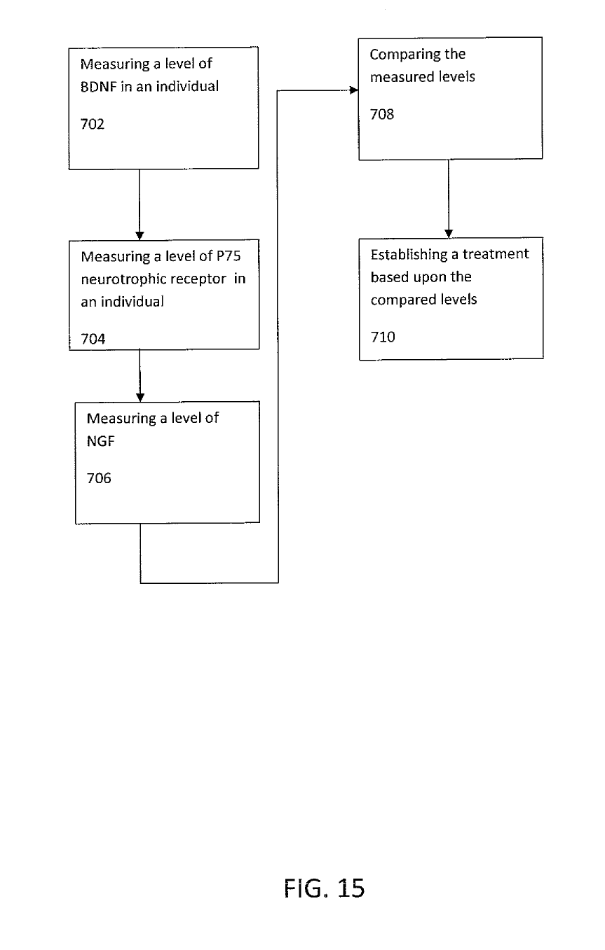

FIG. 15 shows the method of evaluating the homeostatic levels of the modulated pairs in order to provide a base index for administering medication and performing analysis. In step 702, the level of BDNF 52 is measured in an individual. The level of BDNF 52 may be measured in the same manner as the substances discussed above.

The measured level of BDNF 52 in the body or environment may be expressed in various units depending upon the test employed in order to measure the level of the BDNF 52. This level can be expressed by the variable X. There is a preferred range in which X may fall that varies depending upon the type of test that is used in order to measure the level of BDNF 52. In an embodiment of the present invention a standard type of test is used.

In step 704, the level of P75 neurotrophic receptor 58 in an individual is measured. This level can be measured using one of the various processes listed above. The measured level of P75 neurotrophic receptor 58 is preferably expressed in the same units of measurement as that used in the measurement of BDNF 52 so as to more readily compare the levels with respect to each other in order to obtain a base level. This level can be expressed by the variable Y. There is a preferred range in which Y may fall that varies depending upon the type of test that is used in order to measure the level of P75 neurotrophic receptor 58. In an embodiment of the present invention a standard type of test is used, such as a blood test.

In step 706, the level of NGF 59 in an individual is measured. This level can be measured using one of the various processes listed above. The measured level of NGF 59 is preferably expressed in the same units of measurement as that used in the measurement of BDNF 52 so as to more readily compare the levels with respect to each other in order to obtain a base level. This level can be expressed by the variable Z. There is a preferred range in which Z may fall that varies depending upon the type of test that is used in order to measure the level of NGF 59. In an embodiment of the present invention a standard type of test is used.

In step 708, the measured levels X, Y and Z are then compared to standard levels. This may be done in a number of different ways. One possible way in which this can be accomplished is to total the values of all of the levels (X+Y+Z) in order to establish sum level C. This sum level C can be used in step 710 in order to establish a diagnosis of the individual that can be used in determining the provision of medication and other matters related to the measured levels. In particular the levels may be used to determine if more NGF 59 may need to be supplied in order to place the level BDNF 52 or P75 neurotrophic receptor 58 back to proper levels and to further promote neuron synthesis. The NGF 59 may be administered via pill form or some other acceptable means. Alternatively, it may be suggested that an individual's behavior be modified in order to reduce levels of one of the substances.

The assessment of the modulated substances enables the provision of consulting/counseling services relative to the biological foundation for and between the substances associated with neuron synthesis.

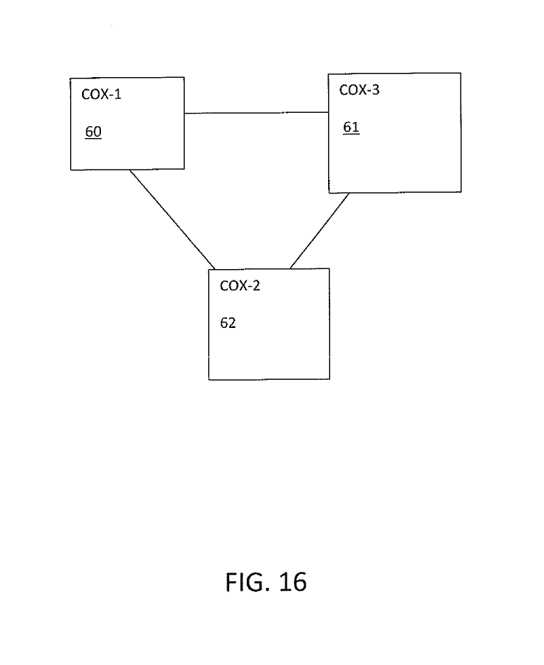

Another example of the determination of homeostatic levels of the sets of modulated pairs discussed above is now provided. The pair of COX-1 60 and COX-3 61 is modulated by COX-2 62 and plays a role in forming enzymes for lipid conversions for smooth muscle tissue. This relationship is shown in FIG. 16.

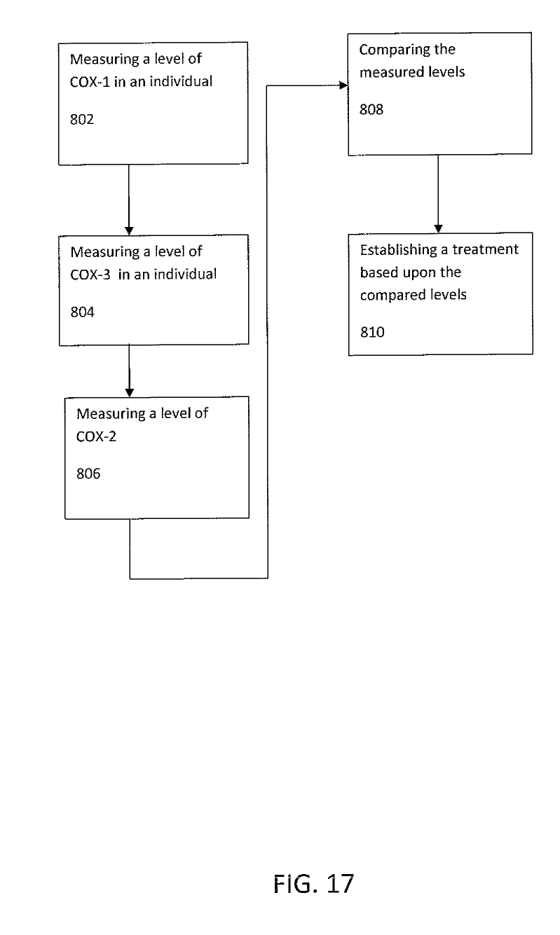

FIG. 17 shows the method of evaluating the homeostatic levels of the modulated pairs in order to provide a base index for administering medication and performing analysis. In step 802, the level of COX-1 60 is measured in an individual. The level of COX-1 60 may be measured in the same manner as the substances discussed above.

The measured level of COX-1 60 in the body or environment may be expressed in various units depending upon the test employed in order to measure the level of the COX-1 60. This level can be expressed by the variable X. There is a preferred range in which X may fall that varies depending upon the type of test that is used in order to measure the level of COX-1 60. In an embodiment of the present invention a standard type of test is used.

In step 804, the level of COX-3 61 in an individual is measured. This level can be measured using one of the various processes listed above. The measured level of COX-3 61 is preferably expressed in the same units of measurement as that used in the measurement of COX-1 60 so as to more readily compare the levels with respect to each other in order to obtain a base level. This level can be expressed by the variable Y. There is a preferred range in which Y may fall that varies depending upon the type of test that is used in order to measure the level of COX-3 61. In an embodiment of the present invention a standard type of test is used, such as a blood test.

In step 806, the level of COX-2 in an individual is measured. This level can be measured using one of the various processes listed above. The measured level of COX-2 62 is preferably expressed in the same units of measurement as that used in the measurement of COX-1 60 so as to more readily compare the levels with respect to each other in order to obtain a base level. This level can be expressed by the variable Z. There is a preferred range in which Z may fall that varies depending upon the type of test that is used in order to measure the level of COX-2 62. In an embodiment of the present invention a standard type of test is used.

In step 808, the measured levels X, Y and Z are then compared to standard levels. This may be done in a number of different ways. One possible way in which this can be accomplished is to total the values of all of the levels (X+Y+Z) in order to establish sum level C. This sum level C can be used in step 810 in order to establish a diagnosis of the individual that can be used in determining the provision of medication and other matters related to the measured levels. In particular the levels may be used to determine if more COX-2 62 may need to be supplied in order to place the level COX-1 60 or COX-3 61 back to proper levels and to further promote neuron synthesis. A value for C that falls below or above the predetermined level may signal the need to administer additional COX-2 62 to the individual. The COX-2 62 may be administered via pill form or some other acceptable means. Alternatively, it may be suggested that an individual's behavior be modified in order to reduce levels of one of the substances.

The assessment of the modulated substances enables the provision of consulting/counseling services relative to the biological foundation for and between the substances associated with enzymes for lipid conversions for smooth muscle tissue.

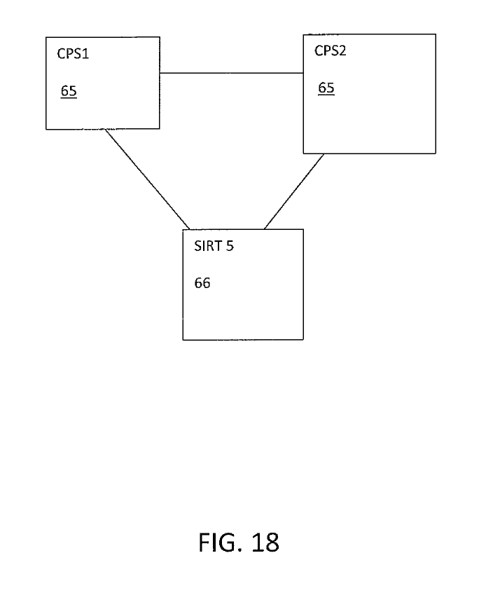

Another example of the determination of homeostatic levels of the sets of modulated pairs discussed above is now discussed. The pair of CPS1 64 and CPS2 65 is modulated by SIRT 5 66 and plays a role in the conversion of pyrimidines. This relationship is shown in FIG. 18.

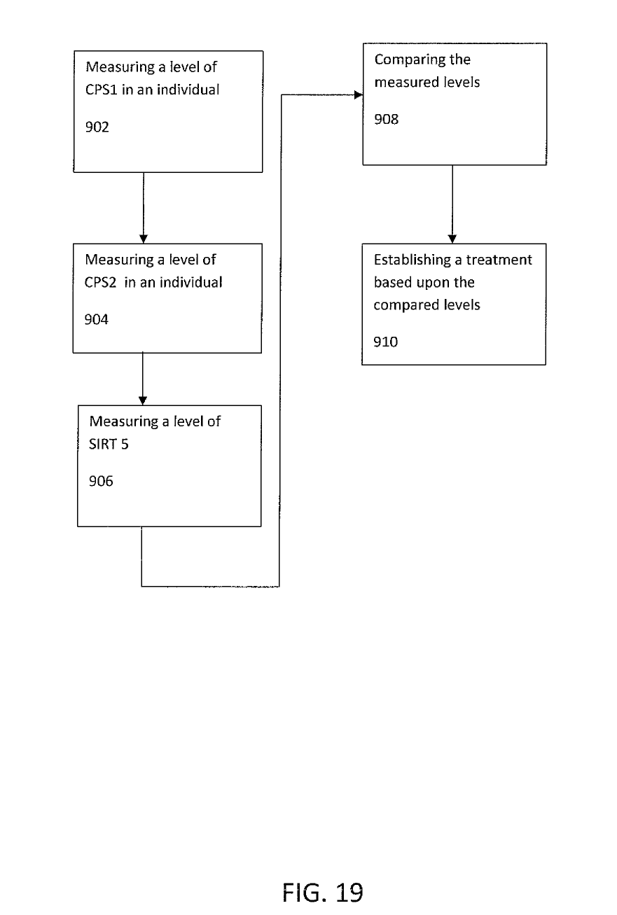

FIG. 19 shows the method of evaluating the homeostatic levels of the modulated pairs in order to provide a base index for administering medication and performing analysis. In step 902, the level of CPS1 64 is measured in an individual. The level of CPS1 64 may be measured in the same manner as the substances discussed above.

The measured level of CPS1 64 in the body or environment may be expressed in various units depending upon the test employed in order to measure the level of the CPS1 64. This level can be expressed by the variable X. There is a preferred range in which X may fall that varies depending upon the type of test that is used in order to measure the level of CPS1 64. In an embodiment of the present invention a standard type of test is used.

In step 904, the level of CPS2 65 in an individual is measured. This level can be measured using one of the various processes listed above. The measured level of CPS2 65 is preferably expressed in the same units of measurement as that used in the measurement of CPS1 64 so as to more readily compare the levels with respect to each other in order to obtain a base level. This level can be expressed by the variable Y. There is a preferred range in which Y may fall that varies depending upon the type of test that is used in order to measure the level of CPS2 65. In an embodiment of the present invention a standard type of test is used, such as a blood test.

In step 906, the level of SIRT 5 66 in an individual is measured. This level can be measured using one of the various processes listed above. The measured level of SIRT 5 66 is preferably expressed in the same units of measurement as that used in the measurement of CPS1 64 so as to more readily compare the levels with respect to each other in order to obtain a base level. This level can be expressed by the variable Z. There is a preferred range in which Z may fall that varies depending upon the type of test that is used in order to measure the level of SIRT 5 66. In an embodiment of the present invention a standard type of test is used.

In step 908, the measured levels X, Y and Z are then compared to standard levels. This may be done in a number of different ways. One possible way in which this can be accomplished is to total the values of all of the levels (X+Y+Z) in order to establish sum level C. This sum level C can be used in step 910 in order to establish a diagnosis of the individual that can be used in determining the provision of medication and other matters related to the measured levels. In particular the levels may be used to determine if more SIRT 5 66 may need to be supplied in order to place the level CPS1 64 or CPS2 65 back to proper levels and to further promote neuron synthesis. A value for C that falls below or above the predetermined level may signal the need to administer additional SIRT 5 65 to the individual. The SIRT 5 65 may be administered via pill form or some other acceptable means. Alternatively, it may be suggested that an individual's behavior be modified in order to reduce levels of one of the substances.

The assessment of the modulated substances enables the provision of consulting/counseling services relative to the biological foundation for and between the substances associated in the conversion of pyrimidines.

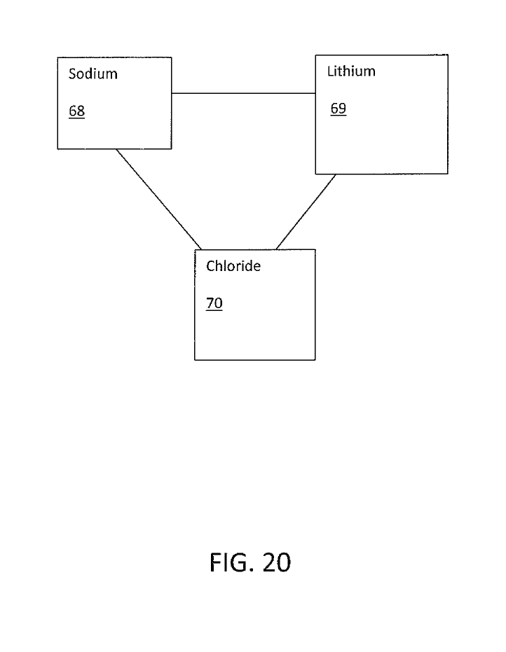

Another example of the determination of homeostatic levels of the sets of modulated pairs discussed above is now discussed. The pair of Na 68 and Li 69 is modulated by chloride 70 and plays a role in the formation of the signaling molecule for oxytocin to cortisol. This relationship is shown in FIG. 20.

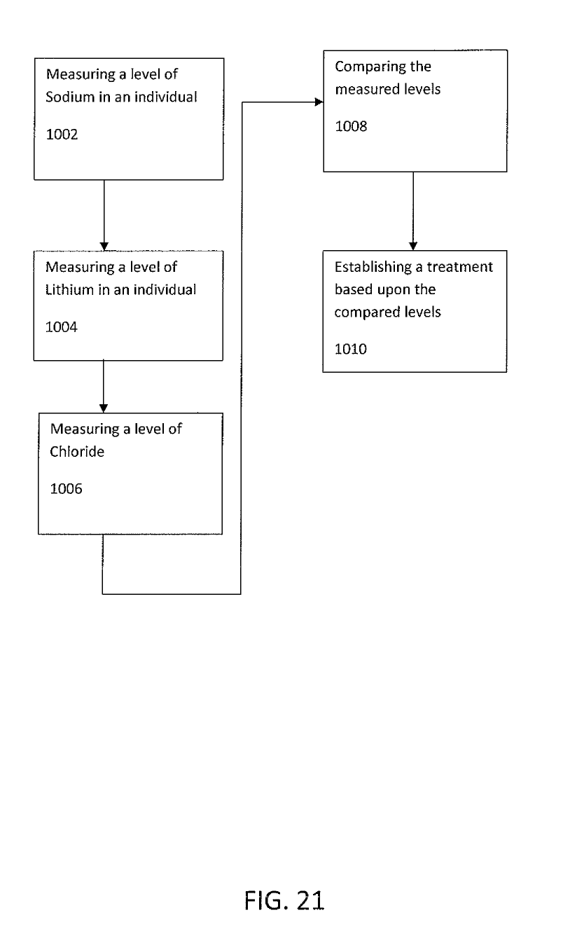

FIG. 21 shows the method of evaluating the homeostatic levels of the modulated pairs in order to provide a base index for administering medication and performing analysis. In step 1002, the level of Na 68 is measured in an individual. The level of Na 68 may be measured in the same manner as the substances discussed above.

The measured level of Na 68 in the body or environment may be expressed in various units depending upon the test employed in order to measure the level of the Na 68. This level can be expressed by the variable X. There is a preferred range in which X may fall that varies depending upon the type of test that is used in order to measure the level of Na 68. In an embodiment of the present invention a standard type of test is used.

In step 1004, the level of Li 69 in an individual is measured. This level can be measured using one of the various processes listed above. The measured level of Li 69 is preferably expressed in the same units of measurement as that used in the measurement of Na 68 so as to more readily compare the levels with respect to each other in order to obtain a base level. This level can be expressed by the variable Y. There is a preferred range in which Y may fall that varies depending upon the type of test that is used in order to measure the level of Li 69. In an embodiment of the present invention a standard type of test is used, such as a blood test.

In step 1006, the level of chloride 70 in an individual is measured. This level can be measured using one of the various processes listed above. The measured level of chloride 70 is preferably expressed in the same units of measurement as that used in the measurement of Na 68 so as to more readily compare the levels with respect to each other in order to obtain a base level. This level can be expressed by the variable Z. There is a preferred range in which Z may fall that varies depending upon the type of test that is used in order to measure the level of chloride 70. In an embodiment of the present invention a standard type of test is used.

In step 1008, the measured levels X, Y and Z are then compared to standard levels. This may be done in a number of different ways. One possible way in which this can be accomplished is to total the values of all of the levels (X+Y+Z) in order to establish sum level C. This sum level C can be used in step 1010 in order to establish a diagnosis of the individual that can be used in determining the provision of medication and other matters related to the measured levels. In particular the levels may be used to determine if more chloride 70 may need to be supplied in order to place the level Na 68 or Li 69 back to proper levels and to further promote neuron synthesis. A value for C that falls below or above the predetermined level may signal the need to administer additional chloride 70 to the individual. The chloride 70 may be administered via pill form or some other acceptable means. Alternatively, it may be suggested that an individual's behavior be modified in order to reduce levels of one of the substances.

The assessment of the modulated substances enables the provision of consulting/counseling services relative to the biological foundation for and between the substances associated in the formation of the signaling molecule for oxytocin to cortisol.

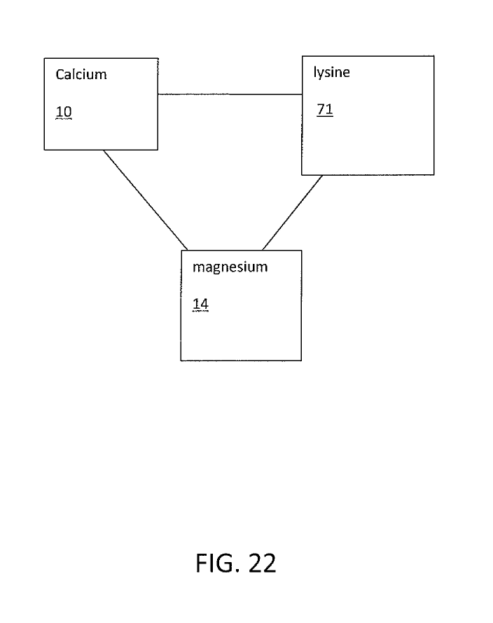

Another example of the determination of homeostatic levels of the sets of modulated pairs discussed above is now discussed. The pair of calcium 10 and lysine 71 is modulated by magnesium 14 and plays a role in the formation of the constituents of calrecticulin. This relationship is shown in FIG. 22.

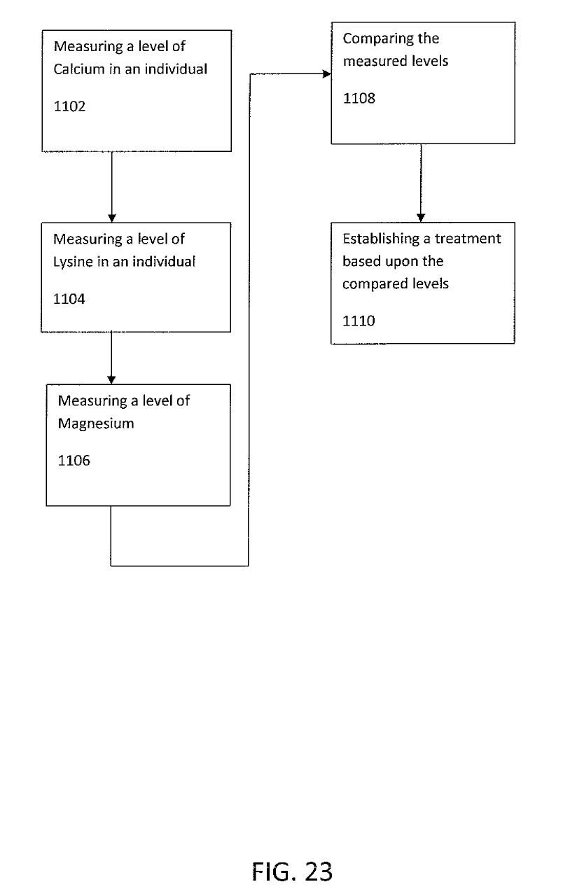

FIG. 23 shows the method of evaluating the homeostatic levels of the modulated pairs in order to provide a base index for administering medication and performing analysis. In step 1102, the level of calcium 10 is measured in an individual. The level of calcium 10 may be measured in the same manner as the substances discussed above.

The measured level of calcium 10 in the body or environment may be expressed in various units depending upon the test employed in order to measure the level of the calcium 10. This level can be expressed by the variable X. There is a preferred range in which X may fall that varies depending upon the type of test that is used in order to measure the level of calcium 10. In an embodiment of the present invention a standard type of test is used.

In step 1104, the level of lysine 71 in an individual is measured. This level can be measured using one of the various processes listed above. The measured level of lysine 71 is preferably expressed in the same units of measurement as that used in the measurement of calcium 10 so as to more readily compare the levels with respect to each other in order to obtain a base level. This level can be expressed by the variable Y. There is a preferred range in which Y may fall that varies depending upon the type of test that is used in order to measure the level of lysine 71. In an embodiment of the present invention a standard type of test is used, such as a blood test.

In step 1106, the level of magnesium 14 in an individual is measured. This level can be measured using one of the various processes listed above. The measured level of magnesium 14 is preferably expressed in the same units of measurement as that used in the measurement of calcium 10 so as to more readily compare the levels with respect to each other in order to obtain a base level. This level can be expressed by the variable Z. There is a preferred range in which Z may fall that varies depending upon the type of test that is used in order to measure the level of Z. In an embodiment of the present invention a standard type of test is used.

In step 1108, the measured levels X, Y and Z are then compared to standard levels. This may be done in a number of different ways. One possible way in which this can be accomplished is to total the values of all of the levels (X+Y+Z) in order to establish sum level C. This sum level C can be used in step 1110 in order to establish a diagnosis of the individual that can be used in determining the provision of medication and other matters related to the measured levels. In particular the levels may be used to determine if more magnesium 14 may need to be supplied in order to place the level calcium 10 or lysine 71 back to proper levels and to further promote neuron synthesis. A value for C that falls below or above the predetermined level may signal the need to administer additional magnesium 14 to the individual. The magnesium 14 may be administered via pill form or some other acceptable means. Alternatively, it may be suggested that an individual's behavior be modified in order to reduce levels of one of the substances.

The assessment of the modulated substances enables the provision of consulting/counseling services relative to the biological foundation for and between the substances associated in the formation of the constituents of calrecticulin.

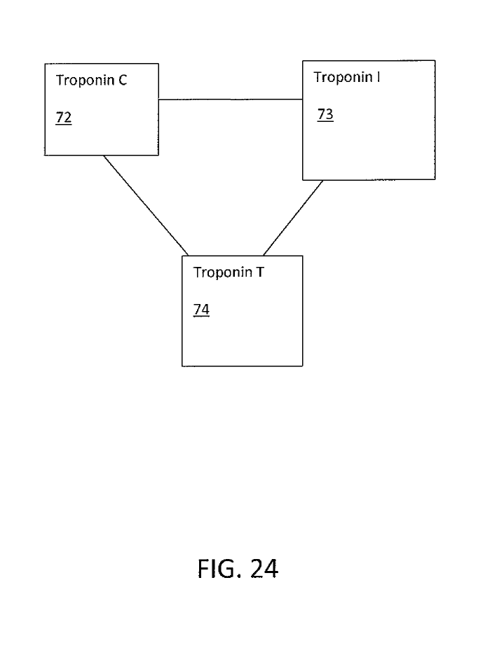

Another example of the determination of homeostatic levels of the sets of modulated pairs discussed above is now discussed. The pair of troponin C 72 and troponin I 73 is modulated by troponin T 74 and plays a role in the formation of the constituents of troponin necessary for homeostasis of muscle contraction. This relationship is shown in FIG. 24.

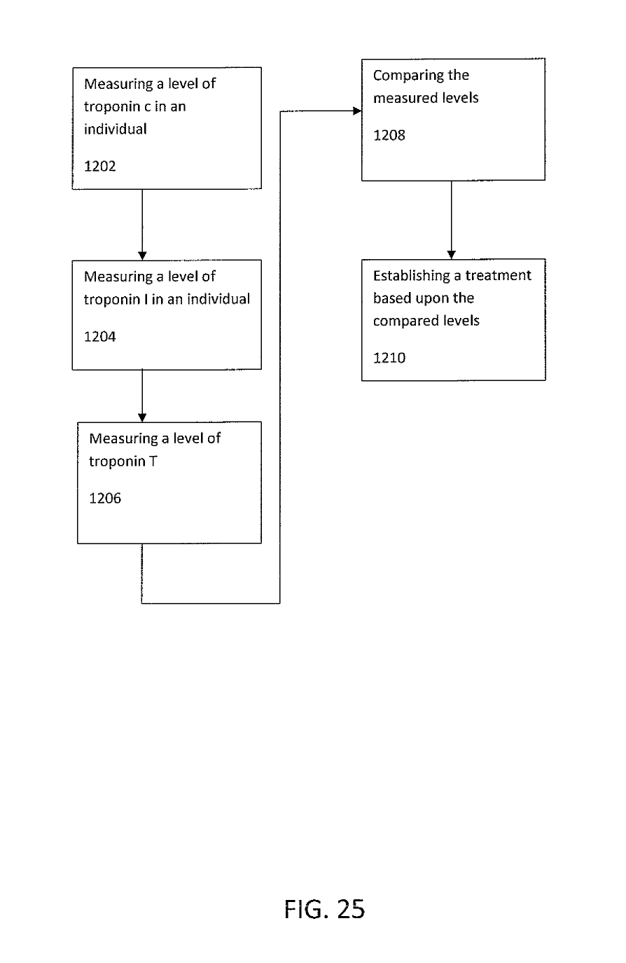

FIG. 25 shows the method of evaluating the homeostatic levels of the modulated pairs in order to provide a base index for administering medication and performing analysis. In step 1202, the level of troponin C 72 is measured in an individual. The level of troponin C 72 may be measured in the same manner as the substances discussed above.

The measured level of troponin C 72 in the body or environment may be expressed in various units depending upon the test employed in order to measure the level of the troponin C 72. This level can be expressed by the variable X. There is a preferred range in which X may fall that varies depending upon the type of test that is used in order to measure the level of troponin C 72. In an embodiment of the present invention a standard type of test is used.

In step 1204, the level of troponin I 73 in an individual is measured. This level can be measured using one of the various processes listed above. The measured level of troponin I 73 is preferably expressed in the same units of measurement as that used in the measurement of troponin C 72 so as to more readily compare the levels with respect to each other in order to obtain a base level. This level can be expressed by the variable Y. There is a preferred range in which Y may fall that varies depending upon the type of test that is used in order to measure the level of troponin I 73. In an embodiment of the present invention a standard type of test is used, such as a blood test.

In step 1206, the level of troponin T 74 in an individual is measured. This level can be measured using one of the various processes listed above. The measured level of troponin T 74 is preferably expressed in the same units of measurement as that used in the measurement of troponin C 72 so as to more readily compare the levels with respect to each other in order to obtain a base level. This level can be expressed by the variable Z. There is a preferred range in which Z may fall that varies depending upon the type of test that is used in order to measure the level of Z. In an embodiment of the present invention a standard type of test is used.

In step 1208, the measured levels X, Y and Z are then compared to standard levels. This may be done in a number of different ways. One possible way in which this can be accomplished is to total the values of all of the levels (X+Y+Z) in order to establish sum level C. This sum level C can be used in step 1210 in order to establish a diagnosis of the individual that can be used in determining the provision of medication and other matters related to the measured levels. In particular the levels may be used to determine if more troponin T 74 may need to be supplied in order to place the level troponin C 72 or troponin I 73 back to proper levels and to further promote neuron synthesis. A value for C that falls below or above the predetermined level may signal the need to administer additional troponin T 74 to the individual. The troponin T 74 may be administered via pill form or some other acceptable means. Alternatively, it may be suggested that an individual's behavior be modified in order to reduce levels of one of the substances.

The assessment of the modulated substances enables the provision of consulting/counseling services relative to the biological foundation for and between the substances associated in the formation of the constituents of troponin necessary for homeostasis of muscle contraction.

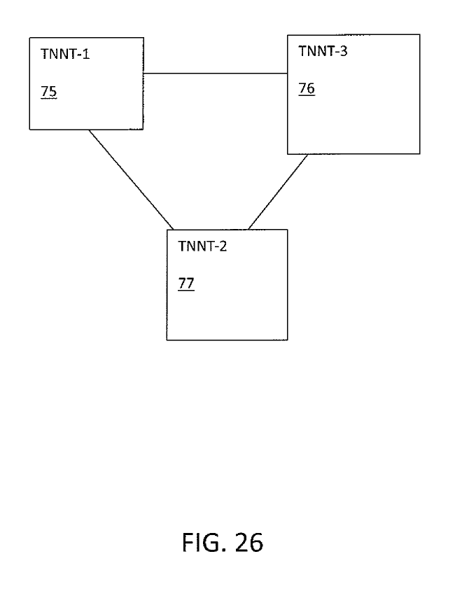

Another example of the determination of homeostatic levels of the sets of modulated pairs discussed above is now discussed. The pair of TNNT-1 75 and TNNT-3 76 is modulated by TNNT-2 77 and plays a role in the formation of troponin T. This relationship is shown in FIG. 26.

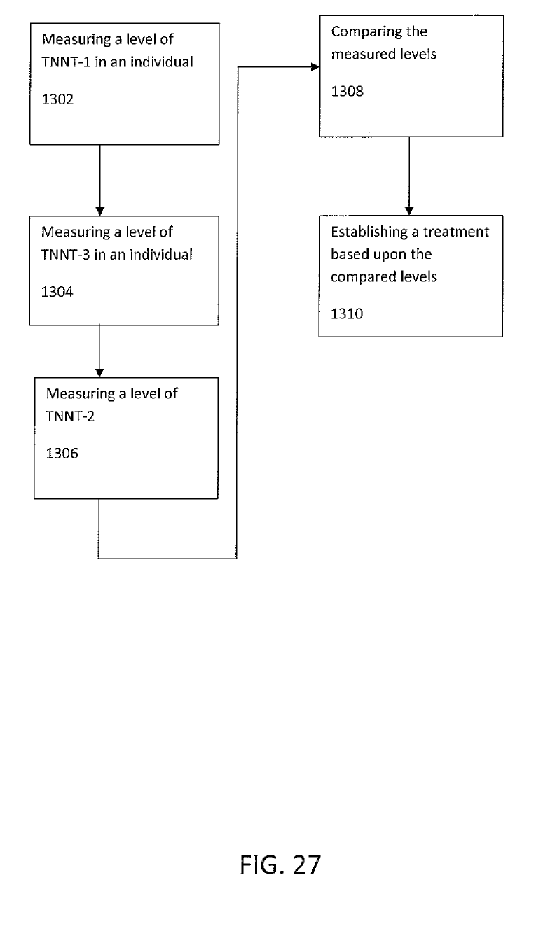

FIG. 27 shows the method of evaluating the homeostatic levels of the modulated pairs in order to provide a base index for administering medication and performing analysis. In step 1302, the level of TNNT-1 75 is measured in an individual. The level of TNNT-1 75 may be measured in the same manner as the substances discussed above.

The measured level of TNNT-1 75 in the body or environment may be expressed in various units depending upon the test employed in order to measure the level of the TNNT-1 75. This level can be expressed by the variable X. There is a preferred range in which X may fall that varies depending upon the type of test that is used in order to measure the level of TNNT-1 75. In an embodiment of the present invention a standard type of test is used.

In step 1304, the level of TNNT-3 76 in an individual is measured. This level can be measured using one of the various processes listed above. The measured level of TNNT-3 76 is preferably expressed in the same units of measurement as that used in the measurement of TNNT-1 75 so as to more readily compare the levels with respect to each other in order to obtain a base level. This level can be expressed by the variable Y. There is a preferred range in which Y may fall that varies depending upon the type of test that is used in order to measure the level of TNNT-3 76. In an embodiment of the present invention a standard type of test is used, such as a blood test.

In step 1306, the level of TNNT-2 77 in an individual is measured. This level can be measured using one of the various processes listed above. The measured level of TNNT-2 77 is preferably expressed in the same units of measurement as that used in the measurement of TNNT-1 75 so as to more readily compare the levels with respect to each other in order to obtain a base level. This level can be expressed by the variable Z. There is a preferred range in which Z may fall that varies depending upon the type of test that is used in order to measure the level of Z. In an embodiment of the present invention a standard type of test is used.

In step 1308, the measured levels X, Y and Z are then compared to standard levels. This may be done in a number of different ways. One possible way in which this can be accomplished is to total the values of all of the levels (X+Y+Z) in order to establish sum level C. This sum level C can be used in step 1310 in order to establish a diagnosis of the individual that can be used in determining the provision of medication and other matters related to the measured levels. In particular the levels may be used to determine if more TNNT-2 77 may need to be supplied in order to place the level of TNNT-1 75 and TNNT-3 76 back to proper levels and to further promote neuron synthesis. A value for C that falls below or above the predetermined level may signal the need to administer additional TNNT-2 77 to the individual. The TNNT-2 77 may be administered via pill form or some other acceptable means. Alternatively, it may be suggested that an individual's behavior be modified in order to reduce levels of one of the substances.

The assessment of the modulated substances enables the provision of consulting/counseling services relative to the biological foundation for and between the substances associated in the formation of troponin T.

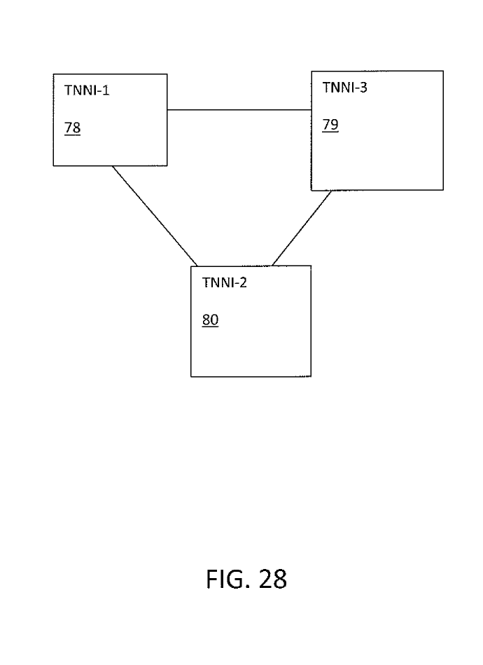

Another example of the determination of homeostatic levels of the sets of modulated pairs discussed above is now discussed. The pair of TNNI-1 78 and TNNI-3 79 is modulated by TNNI-2 80 and plays a role in the formation of troponin I. This relationship is shown in FIG. 28.

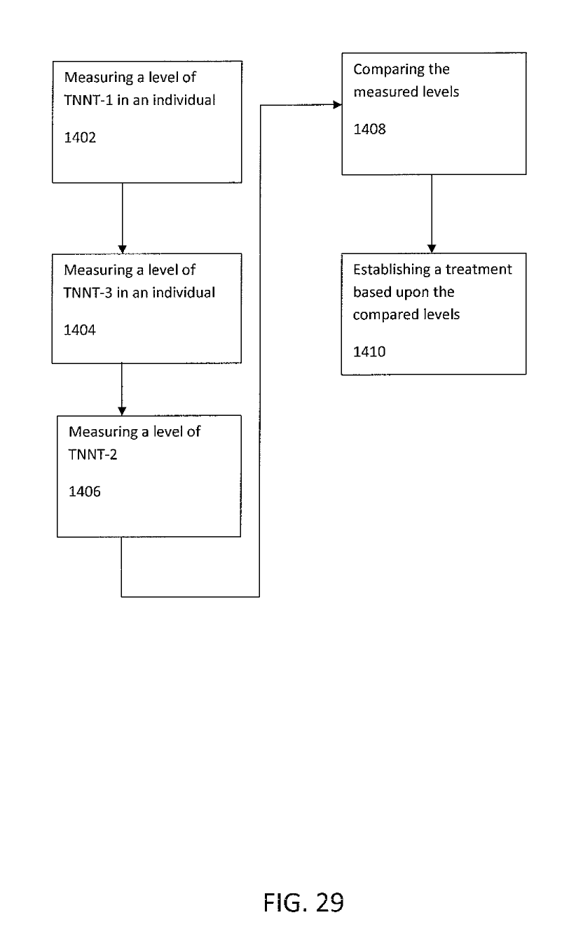

FIG. 29 shows the method of evaluating the homeostatic levels of the modulated pairs in order to provide a base index for administering medication and performing analysis. In step 1402, the level of TNNI-1 78 is measured in an individual. The level of TNNI-1 78 may be measured in the same manner as the substances discussed above.

The measured level of TNNI-1 78 in the body or environment may be expressed in various units depending upon the test employed in order to measure the level of the TNNI-1 78. This level can be expressed by the variable X. There is a preferred range in which X may fall that varies depending upon the type of test that is used in order to measure the level of TNNI-1 78. In an embodiment of the present invention a standard type of test is used.

In step 1404, the level of TNNI-3 79 in an individual is measured. This level can be measured using one of the various processes listed above. The measured level of TNNI-3 79 is preferably expressed in the same units of measurement as that used in the measurement of TNNI-1 78 so as to more readily compare the levels with respect to each other in order to obtain a base level. This level can be expressed by the variable Y. There is a preferred range in which Y may fall that varies depending upon the type of test that is used in order to measure the level of TNNI-3 79. In an embodiment of the present invention a standard type of test is used, such as a blood test.

In step 1406, the level of TNNI-2 80 in an individual is measured. This level can be measured using one of the various processes listed above. The measured level of TNNI-2 80 is preferably expressed in the same units of measurement as that used in the measurement of TNNI-1 78 so as to more readily compare the levels with respect to each other in order to obtain a base level. This level can be expressed by the variable Z. There is a preferred range in which Z may fall that varies depending upon the type of test that is used in order to measure the level of Z. In an embodiment of the present invention a standard type of test is used.

In step 1408, the measured levels X, Y and Z are then compared to standard levels. This may be done in a number of different ways. One possible way in which this can be accomplished is to total the values of all of the levels (X+Y+Z) in order to establish sum level C. This sum level C can be used in step 1410 in order to establish a diagnosis of the individual that can be used in determining the provision of medication and other matters related to the measured levels. In particular the levels may be used to determine if more TNNI-2 80 may need to be supplied in order to place the level of TNNI-1 78 and TNNI-3 79 back to proper levels and to further promote neuron synthesis. A value for C that falls below or above the predetermined level may signal the need to administer additional TNNI-2 80 to the individual. The TNNI-2 80 may be administered via pill form or some other acceptable means. Alternatively, it may be suggested that an individual's behavior be modified in order to reduce levels of one of the substances.

The assessment of the modulated substances enables the provision of consulting/counseling services relative to the biological foundation for and between the substances associated in the formation of troponin I.

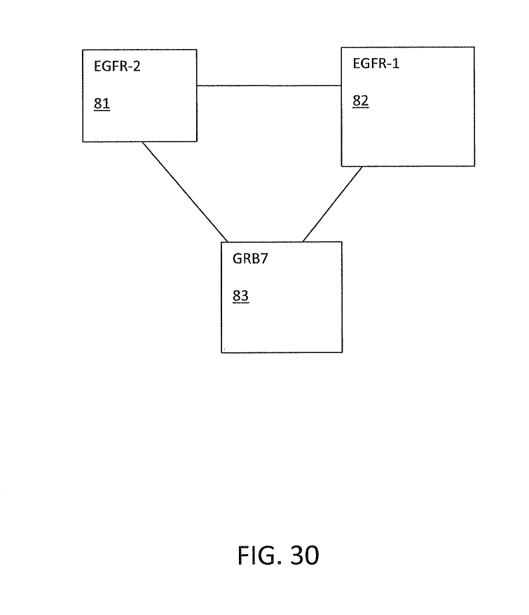

Another example of the determination of homeostatic levels of the sets of modulated pairs discussed above is now discussed. The pair of EGFR-4 81 and EGFR-1 82 is modulated by GRB7 83 and plays a role in factors for cell proliferation and adhesion. This relationship is shown in FIG. 30.

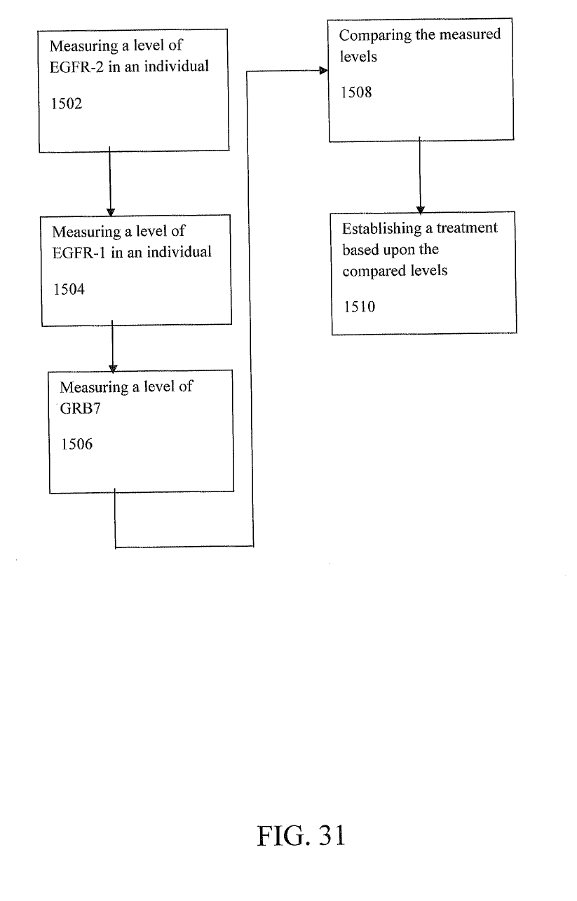

FIG. 31 shows the method of evaluating the homeostatic levels of the modulated pairs in order to provide a base index for administering medication and performing analysis. In step 1502, the level of EGFR-4 81 is measured in an individual. The level of EGFR-4 81 may be measured in the same manner as the substances discussed above.

The measured level of EGFR-4 81 in the body or environment may be expressed in various units depending upon the test employed in order to measure the level of the EGFR-4 81. This level can be expressed by the variable X. There is a preferred range in which X may fall that varies depending upon the type of test that is used in order to measure the level of EGFR-4 81. In an embodiment of the present invention a standard type of test is used.

In step 1504, the level of EGFR-1 82 in an individual is measured. This level can be measured using one of the various processes listed above. The measured level of EGFR-1 82 is preferably expressed in the same units of measurement as that used in the measurement of EGFR-4 81 so as to more readily compare the levels with respect to each other in order to obtain a base level. This level can be expressed by the variable Y. There is a preferred range in which Y may fall that varies depending upon the type of test that is used in order to measure the level of EGFR-1 82. In an embodiment of the present invention a standard type of test is used, such as a blood test.

In step 1506, the level of GRB7 83 in an individual is measured. This level can be measured using one of the various processes listed above. The measured level of GRB7 83 is preferably expressed in the same units of measurement as that used in the measurement of EGFR-4 81 so as to more readily compare the levels with respect to each other in order to obtain a base level. This level can be expressed by the variable Z. There is a preferred range in which Z may fall that varies depending upon the type of test that is used in order to measure the level of Z. In an embodiment of the present invention a standard type of test is used.

In step 1508, the measured levels X, Y and Z are then compared to standard levels. This may be done in a number of different ways. One possible way in which this can be accomplished is to total the values of all of the levels (X+Y+Z) in order to establish sum level C. This sum level C can be used in step 1510 in order to establish a diagnosis of the individual that can be used in determining the provision of medication and other matters related to the measured levels. In particular the levels may be used to determine if more GRB7 83 may need to be supplied in order to place the level of EGFR-4 81 and EGFR-1 82 back to proper levels and to further promote neuron synthesis. A value for C that falls below or above the predetermined level may signal the need to administer additional GRB7 83 to the individual. The GRB7 83 may be administered via pill form or some other acceptable means. Alternatively, it may be suggested that an individual's behavior be modified in order to reduce levels of one of the substances.

The assessment of the modulated substances enables the provision of consulting/counseling services relative to the biological foundation for and between the substances associated in playing a role in factors for cell proliferation and adhesion.

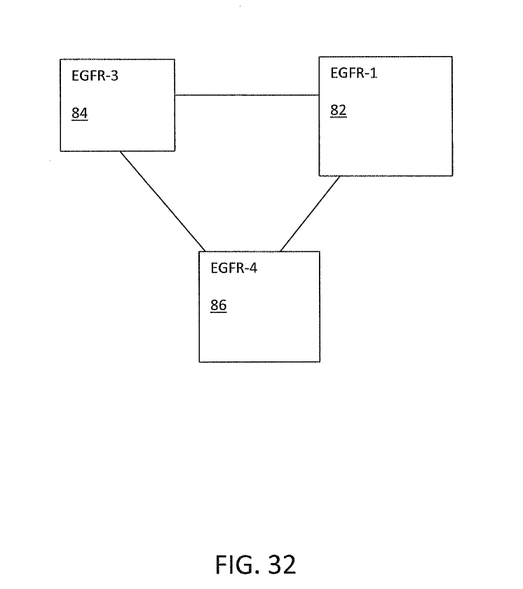

Another example of the determination of homeostatic levels of the sets of modulated pairs discussed above is now discussed. The pair of EGFR-2 84 and EGFR-1 82 is modulated by EGFR-3 86 and plays a role in factors for cell proliferation and differentiation. This relationship is shown in FIG. 32.

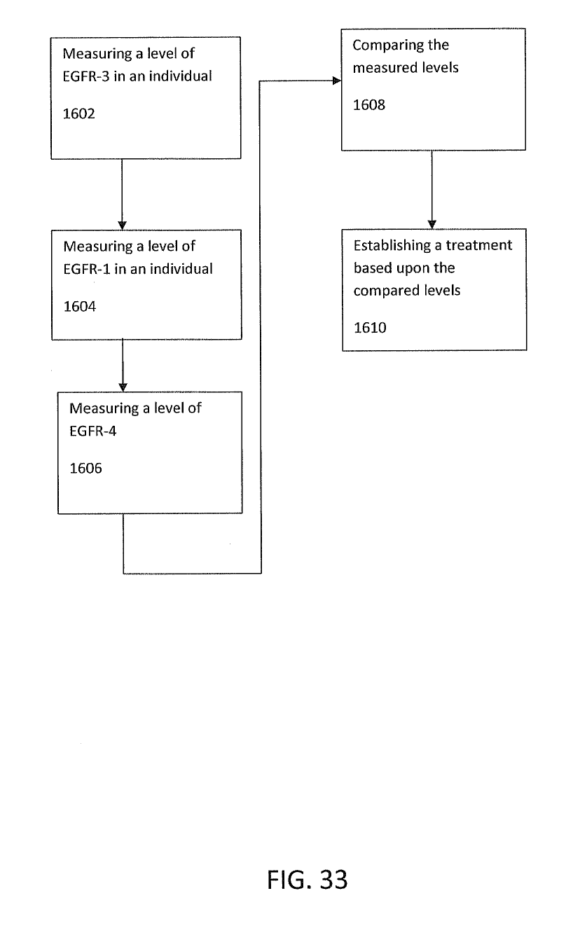

FIG. 33 shows the method of evaluating the homeostatic levels of the modulated pairs in order to provide a base index for administering medication and performing analysis. In step 1602, the level of EGFR-2 84 is measured in an individual. The level of EGFR-2 84 may be measured in the same manner as the substances discussed above.

The measured level of EGFR-2 84 in the body or environment may be expressed in various units depending upon the test employed in order to measure the level of the EGFR-2 84. This level can be expressed by the variable X. There is a preferred range in which X may fall that varies depending upon the type of test that is used in order to measure the level of EGFR-2 84. In an embodiment of the present invention a standard type of test is used.

In step 1604, the level of EGFR-1 82 in an individual is measured. This level can be measured using one of the various processes listed above. The measured level of EGFR-1 82 is preferably expressed in the same units of measurement as that used in the measurement of EGFR-2 84 so as to more readily compare the levels with respect to each other in order to obtain a base level. This level can be expressed by the variable Y. There is a preferred range in which Y may fall that varies depending upon the type of test that is used in order to measure the level of EGFR-1 82. In an embodiment of the present invention a standard type of test is used, such as a blood test.

In step 1606, the level of EGFR-3 86 in an individual is measured. This level can be measured using one of the various processes listed above. The measured level of EGFR-3 86 is preferably expressed in the same units of measurement as that used in the measurement of EGFR-2 84 so as to more readily compare the levels with respect to each other in order to obtain a base level. This level can be expressed by the variable Z. There is a preferred range in which Z may fall that varies depending upon the type of test that is used in order to measure the level of Z. In an embodiment of the present invention a standard type of test is used.

In step 1608, the measured levels X, Y and Z are then compared to standard levels. This may be done in a number of different ways. One possible way in which this can be accomplished is to total the values of all of the levels (X+Y+Z) in order to establish sum level C. This sum level C can be used in step 1610 in order to establish a diagnosis of the individual that can be used in determining the provision of medication and other matters related to the measured levels. In particular the levels may be used to determine if more EGFR-3 86 may need to be supplied in order to place the level of EGFR-2 84 and EGFR-1 82 back to proper levels and to further promote neuron synthesis. A value for C that falls below or above the predetermined level may signal the need to administer additional EGFR-3 86 to the individual. The EGFR-3 86 may be administered via pill form or some other acceptable means. Alternatively, it may be suggested that an individual's behavior be modified in order to reduce levels of one of the substances.

The assessment of the modulated substances enables the provision of consulting/counseling services relative to the biological foundation for and between the substances associated in playing a role in factors for cell proliferation and differentiation.

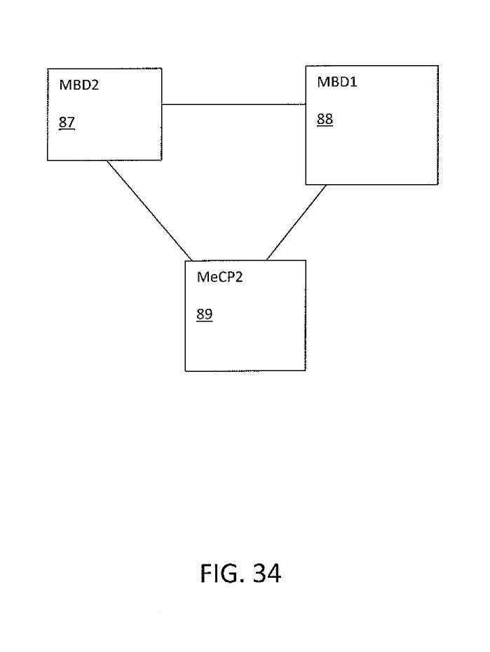

Another example of the determination of homeostatic levels of the sets of modulated pairs discussed above is now discussed. The pair of MBD2 87 and MBD1 88 is modulated by MeCP2 89 and plays a role in factors for the formation constituents of chromatin. This relationship is shown in FIG. 34.

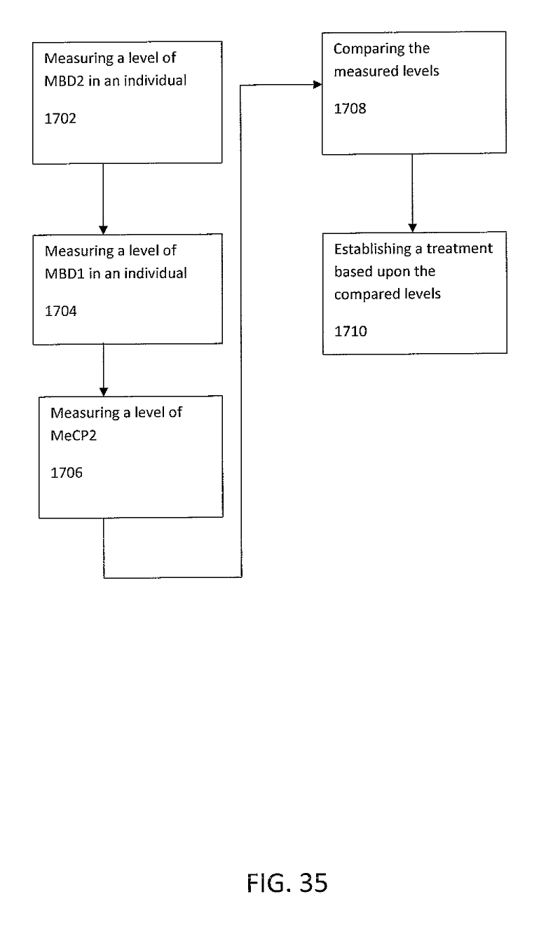

FIG. 35 shows the method of evaluating the homeostatic levels of the modulated pairs in order to provide a base index for administering medication and performing analysis. In step 1702, the level of MBD2 87 is measured in an individual. The level of MBD2 87 may be measured in the same manner as the substances discussed above.

The measured level of MBD2 87 in the body or environment may be expressed in various units depending upon the test employed in order to measure the level of the MBD2 87. This level can be expressed by the variable X. There is a preferred range in which X may fall that varies depending upon the type of test that is used in order to measure the level of MBD2 87. In an embodiment of the present invention a standard type of test is used.

In step 1704, the level of MBD1 88 in an individual is measured. This level can be measured using one of the various processes listed above. The measured level of MBD1 88 is preferably expressed in the same units of measurement as that used in the measurement of MBD2 87 so as to more readily compare the levels with respect to each other in order to obtain a base level. This level can be expressed by the variable Y. There is a preferred range in which Y may fall that varies depending upon the type of test that is used in order to measure the level of MBD1 88. In an embodiment of the present invention a standard type of test is used, such as a blood test.

In step 1706, the level of MeCP2 89 in an individual is measured. This level can be measured using one of the various processes listed above. The measured level of MeCP2 89 is preferably expressed in the same units of measurement as that used in the measurement of MBD2 87 so as to more readily compare the levels with respect to each other in order to obtain a base level. This level can be expressed by the variable Z. There is a preferred range in which Z may fall that varies depending upon the type of test that is used in order to measure the level of Z. In an embodiment of the present invention a standard type of test is used.

In step 1708, the measured levels X, Y and Z are then compared to standard levels. This may be done in a number of different ways. One possible way in which this can be accomplished is to total the values of all of the levels (X+Y+Z) in order to establish sum level C. This sum level C can be used in step 1710 in order to establish a diagnosis of the individual that can be used in determining the provision of medication and other matters related to the measured levels. In particular the levels may be used to determine if more MeCP2 89 may need to be supplied in order to place the level of MBD2 87 and MBD1 88 back to proper levels and to further promote neuron synthesis. A value for C that falls below or above the predetermined level may signal the need to administer additional MeCP2 89 to the individual. The MeCP2 89 may be administered via pill form or some other acceptable means. Alternatively, it may be suggested that an individual's behavior be modified in order to reduce levels of one of the substances.

The assessment of the modulated substances enables the provision of consulting/counseling services relative to the biological foundation for and between the substances associated in formation of the constituents of chromatin.

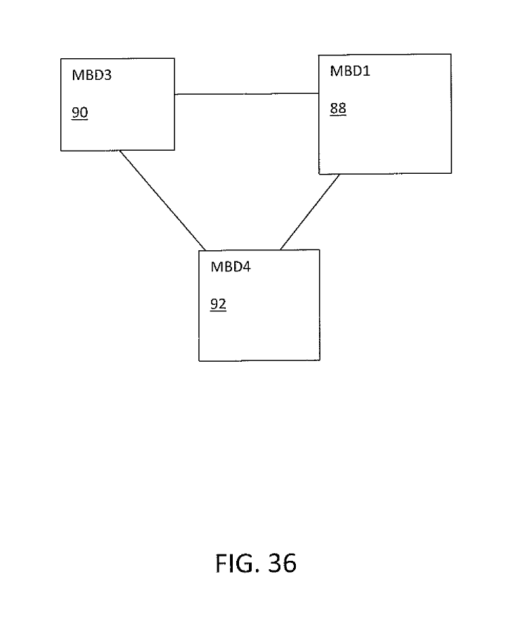

Another example of the determination of homeostatic levels of the sets of modulated pairs discussed above is now discussed. The pair of MBD3 90 and MBD1 88 is modulated by MBD4 92 and plays a role in factors for the formation constituents of chromatin. This relationship is shown in FIG. 36.

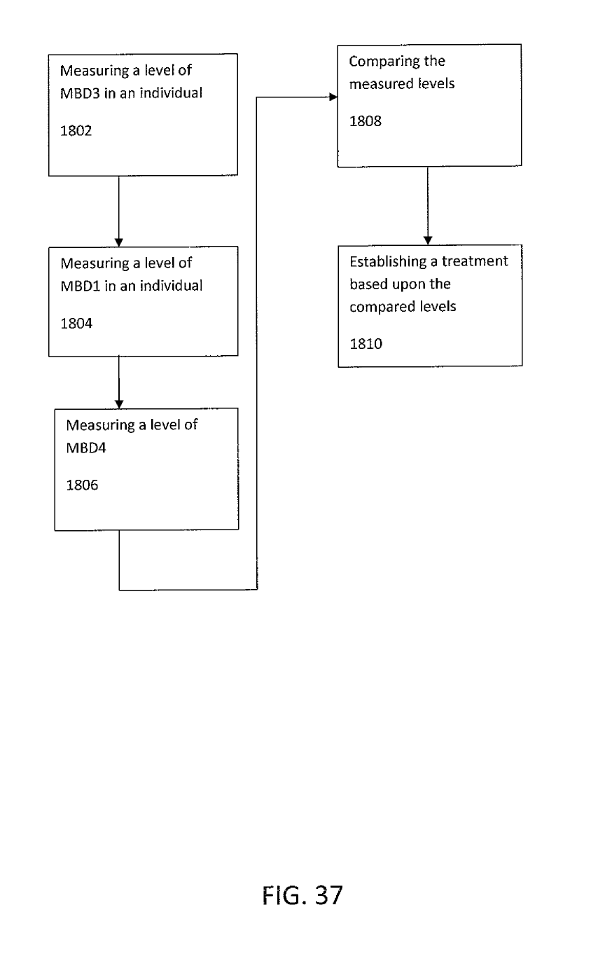

FIG. 37 shows the method of evaluating the homeostatic levels of the modulated pairs in order to provide a base index for administering medication and performing analysis. In step 1802, the level of MBD3 90 is measured in an individual. The level of MBD3 90 may be measured in the same manner as the substances discussed above.

The measured level of MBD3 90 in the body or environment may be expressed in various units depending upon the test employed in order to measure the level of the MBD3 90. This level can be expressed by the variable X. There is a preferred range in which X may fall that varies depending upon the type of test that is used in order to measure the level of MBD3 90. In an embodiment of the present invention a standard type of test is used.

In step 1804, the level of MBD1 88 in an individual is measured. This level can be measured using one of the various processes listed above. The measured level of MBD1 88 is preferably expressed in the same units of measurement as that used in the measurement of MBD3 90 so as to more readily compare the levels with respect to each other in order to obtain a base level. This level can be expressed by the variable Y. There is a preferred range in which Y may fall that varies depending upon the type of test that is used in order to measure the level of MBD1 88. In an embodiment of the present invention a standard type of test is used, such as a blood test.

In step 1806, the level of MBD4 92 in an individual is measured. This level can be measured using one of the various processes listed above. The measured level of MBD4 92 is preferably expressed in the same units of measurement as that used in the measurement of MBD3 90 so as to more readily compare the levels with respect to each other in order to obtain a base level. This level can be expressed by the variable Z. There is a preferred range in which Z may fall that varies depending upon the type of test that is used in order to measure the level of Z. In an embodiment of the present invention a standard type of test is used.

In step 1808, the measured levels X, Y and Z are then compared to standard levels. This may be done in a number of different ways. One possible way in which this can be accomplished is to total the values of all of the levels (X+Y+Z) in order to establish sum level C. This sum level C can be used in step 1810 in order to establish a diagnosis of the individual that can be used in determining the provision of medication and other matters related to the measured levels. In particular the levels may be used to determine if more MBD4 92 may need to be supplied in order to place the level of MBD3 90 and MBD1 88 back to proper levels and to further promote neuron synthesis. A value for C that falls below or above the predetermined level may signal the need to administer additional MBD4 92 to the individual. The MBD4 92 may be administered via pill form or some other acceptable means. Alternatively, it may be suggested that an individual's behavior be modified in order to reduce levels of one of the substances.

The assessment of the modulated substances enables the provision of consulting/counseling services relative to the biological foundation for and between the substances associated in formation of the protein constituents for the maintenance of chromatin.

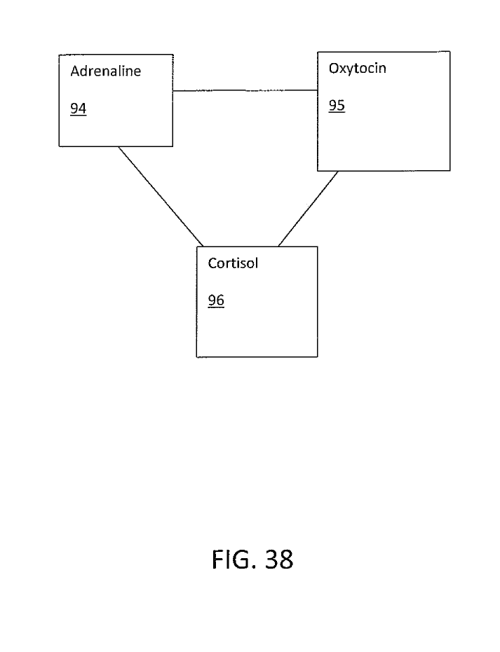

Another example of the determination of homeostatic levels of the sets of modulated pairs discussed above is now discussed. The pair of adrenaline 94 and oxytocin 95 is modulated by cortisol 96 and plays a role in regulating anger and contentment. This relationship is shown in FIG. 38.

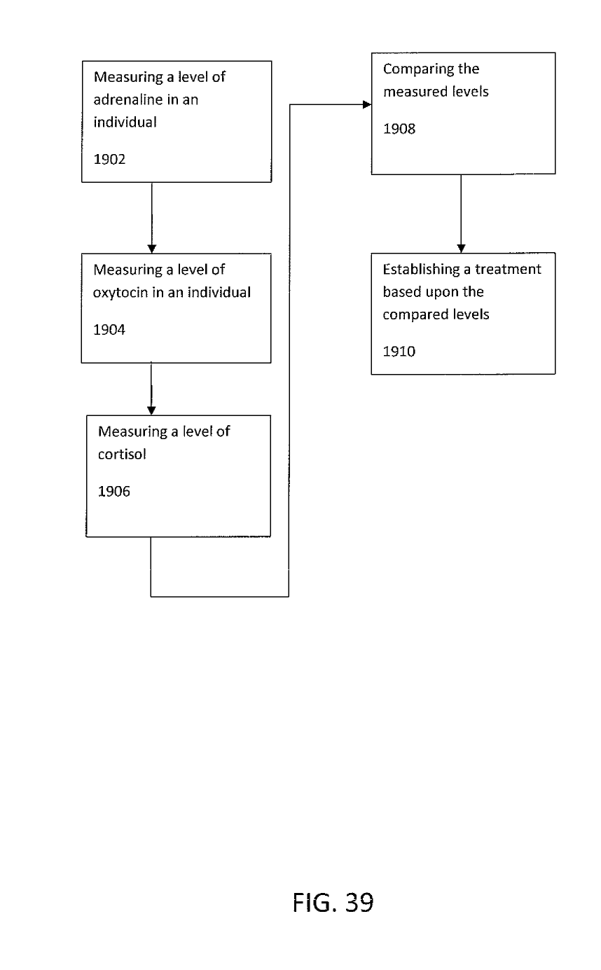

FIG. 39 shows the method of evaluating the homeostatic levels of the modulated pairs in order to provide a base index for administering medication and performing analysis. In step 1902, the level of adrenaline 94 is measured in an individual. The level of adrenaline 94 may be measured in the same manner as the substances discussed above.

The measured level of adrenaline 94 in the body or environment may be expressed in various units depending upon the test employed in order to measure the level of the adrenaline 94. This level can be expressed by the variable X. There is a preferred range in which X may fall that varies depending upon the type of test that is used in order to measure the level of adrenaline 94. In an embodiment of the present invention a standard type of test is used.

In step 1904, the level of oxytocin 95 in an individual is measured. This level can be measured using one of the various processes listed above. The measured level of oxytocin 95 is preferably expressed in the same units of measurement as that used in the measurement of adrenaline 94 so as to more readily compare the levels with respect to each other in order to obtain a base level. This level can be expressed by the variable Y. There is a preferred range in which Y may fall that varies depending upon the type of test that is used in order to measure the level of oxytocin 95. In an embodiment of the present invention a standard type of test is used, such as a blood test.

In step 1906, the level of cortisol 96 in an individual is measured. This level can be measured using one of the various processes listed above. The measured level of cortisol 96 is preferably expressed in the same units of measurement as that used in the measurement of adrenaline 94 so as to more readily compare the levels with respect to each other in order to obtain a base level. This level can be expressed by the variable Z. There is a preferred range in which Z may fall that varies depending upon the type of test that is used in order to measure the level of Z. In an embodiment of the present invention a standard type of test is used.

In step 1908, the measured levels X, Y and Z are then compared to standard levels. This may be done in a number of different ways. One possible way in which this can be accomplished is to total the values of all of the levels (X+Y+Z) in order to establish sum level C. This sum level C can be used in step 1910 in order to establish a diagnosis of the individual that can be used in determining the provision of medication and other matters related to the measured levels. In particular the levels may be used to determine if more cortisol 96 may need to be supplied in order to place the level of adrenaline 94 and oxytocin 95 back to proper levels and to further promote neuron synthesis. A value for C that falls below or above the predetermined level may signal the need to administer additional cortisol 96 to the individual. The cortisol 96 may be administered via pill form or some other acceptable means. Alternatively, it may be suggested that an individual's behavior be modified in order to reduce levels of one of the substances.

The assessment of the modulated substances enables the provision of consulting/counseling services relative to the biological foundation for and between the substances associated regulating anger and contentment.

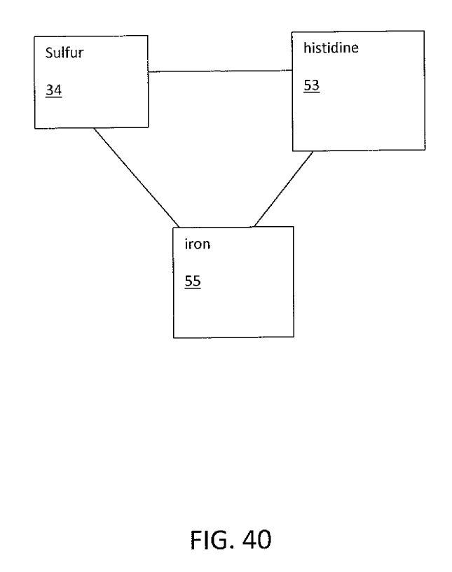

Another example of the determination of homeostatic levels of the sets of modulated pairs discussed above is now discussed. The pair of sulfur 34 and histidine 53 is modulated by iron 55 and playing a role in the process for the formation of Histidine-rich Glycoprotein (HRG). This relationship is shown in FIG. 40.

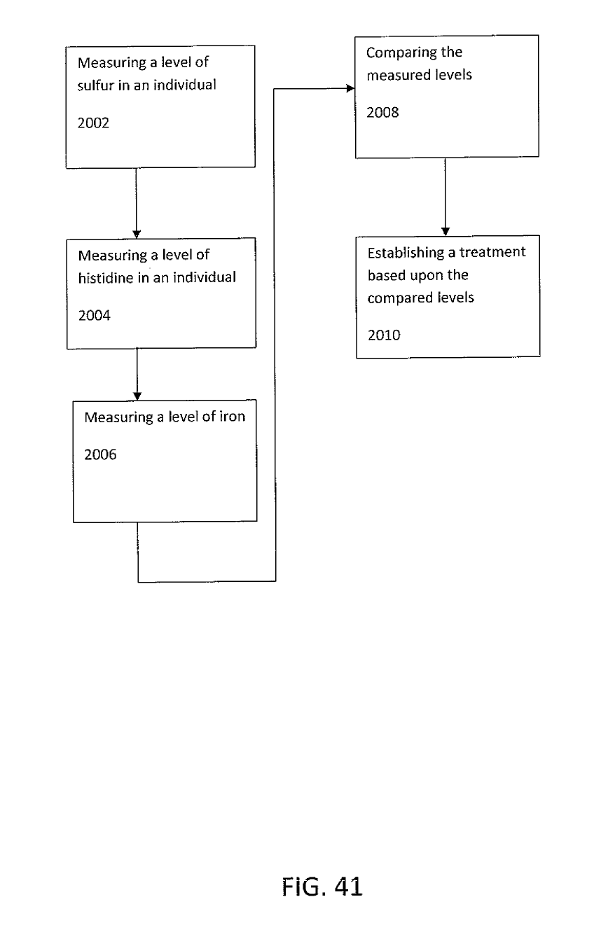

FIG. 41 shows the method of evaluating the homeostatic levels of the modulated pairs in order to provide a base index for administering medication and performing analysis. In step 2002, the level of sulfur 34 is measured in an individual. The level of sulfur 34 may be measured in the same manner as the substances discussed above.

The measured level of sulfur 34 in the body or environment may be expressed in various units depending upon the test employed in order to measure the level of the sulfur 34. This level can be expressed by the variable X. There is a preferred range in which X may fall that varies depending upon the type of test that is used in order to measure the level of sulfur 34. In an embodiment of the present invention a standard type of test is used.

In step 2004, the level of histidine 53 in an individual is measured. This level can be measured using one of the various processes listed above. The measured level of histidine 53 is preferably expressed in the same units of measurement as that used in the measurement of sulfur 34 so as to more readily compare the levels with respect to each other in order to obtain a base level. This level can be expressed by the variable Y. There is a preferred range in which Y may fall that varies depending upon the type of test that is used in order to measure the level of histidine 53. In an embodiment of the present invention a standard type of test is used, such as a blood test.

In step 2006, the level of iron 55 in an individual is measured. This level can be measured using one of the various processes listed above. The measured level of iron 55 is preferably expressed in the same units of measurement as that used in the measurement of sulfur 34 so as to more readily compare the levels with respect to each other in order to obtain a base level. This level can be expressed by the variable Z. There is a preferred range in which Z may fall that varies depending upon the type of test that is used in order to measure the level of Z. In an embodiment of the present invention a standard type of test is used.

In step 2008, the measured levels X, Y and Z are then compared to standard levels. This may be done in a number of different ways. One possible way in which this can be accomplished is to total the values of all of the levels (X+Y+Z) in order to establish sum level C. This sum level C can be used in step 2010 in order to establish a diagnosis of the individual that can be used in determining the provision of medication and other matters related to the measured levels. In particular the levels may be used to determine if more iron 55 may need to be supplied in order to place the level of sulfur 34 and histidine 53 back to proper levels and to further promote neuron synthesis. A value for C that falls below or above the predetermined level may signal the need to administer additional iron 55 to the individual. The iron 55 may be administered via pill form or some other acceptable means. Alternatively, it may be suggested that an individual's behavior be modified in order to reduce levels of one of the substances.

The assessment of the modulated substances enables the provision of consulting/counseling services relative to the biological foundation for and between the substances associated in playing a role in the process for the formation of Histidine-rich Glycoprotein (HRG).

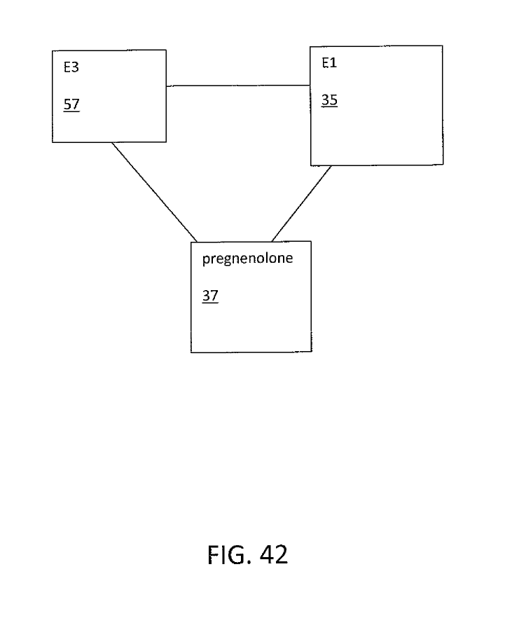

Another example of the determination of homeostatic levels of the sets of modulated pairs discussed above is now discussed. The pair of E3 57 and E1 35 is modulated by pregnenolone 37 and plays a role in the formation of Estrogen. This relationship is shown in FIG. 42.

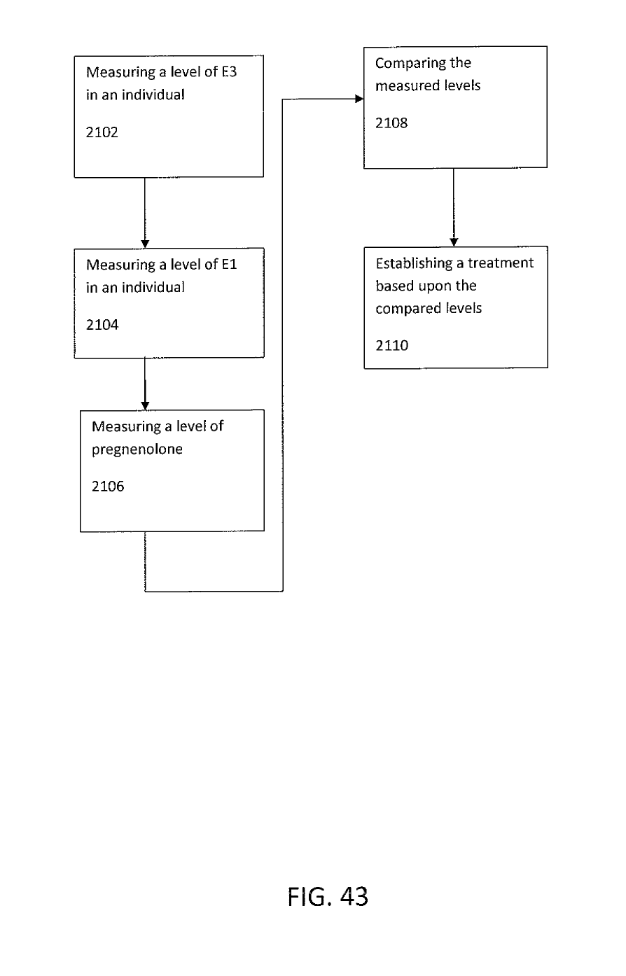

FIG. 43 shows the method of evaluating the homeostatic levels of the modulated pairs in order to provide a base index for administering medication and performing analysis. In step 2102, the level of E3 57 is measured in an individual. The level of E3 57 may be measured in the same manner as the substances discussed above.

The measured level of E3 57 in the body or environment may be expressed in various units depending upon the test employed in order to measure the level of the E3 57. This level can be expressed by the variable X. There is a preferred range in which X may fall that varies depending upon the type of test that is used in order to measure the level of E3 57. In an embodiment of the present invention a standard type of test is used.

In step 2104, the level of E3 57 in an individual is measured. This level can be measured using one of the various processes listed above. The measured level of E1 35 is preferably expressed in the same units of measurement as that used in the measurement of E3 57 so as to more readily compare the levels with respect to each other in order to obtain a base level. This level can be expressed by the variable Y. There is a preferred range in which Y may fall that varies depending upon the type of test that is used in order to measure the level of E1 35. In an embodiment of the present invention a standard type of test is used, such as a blood test.

In step 2106, the level of pregnenolone 37 in an individual is measured. This level can be measured using one of the various processes listed above. The measured level of pregnenolone 37 is preferably expressed in the same units of measurement as that used in the measurement of E3 57 so as to more readily compare the levels with respect to each other in order to obtain a base level. This level can be expressed by the variable Z. There is a preferred range in which Z may fall that varies depending upon the type of test that is used in order to measure the level of Z. In an embodiment of the present invention a standard type of test is used.

In step 2108, the measured levels X, Y and Z are then compared to standard levels. This may be done in a number of different ways. One possible way in which this can be accomplished is to total the values of all of the levels (X+Y+Z) in order to establish sum level C. This sum level C can be used in step 2110 in order to establish a diagnosis of the individual that can be used in determining the provision of medication and other matters related to the measured levels. In particular the levels may be used to determine if more pregnenolone 37 may need to be supplied in order to place the level of E3 57 and E1 35 back to proper levels and to further promote neuron synthesis. A value for C that falls below or above the predetermined level may signal the need to administer additional pregnenolone 37 to the individual. The pregnenolone 37 may be administered via pill form or some other acceptable means. Alternatively, it may be suggested that an individual's behavior be modified in order to reduce levels of one of the substances.

The assessment of the modulated substances enables the provision of consulting/counseling services relative to the biological foundation for and between the substances associated in playing a role in the process of the formation of Estrogen.

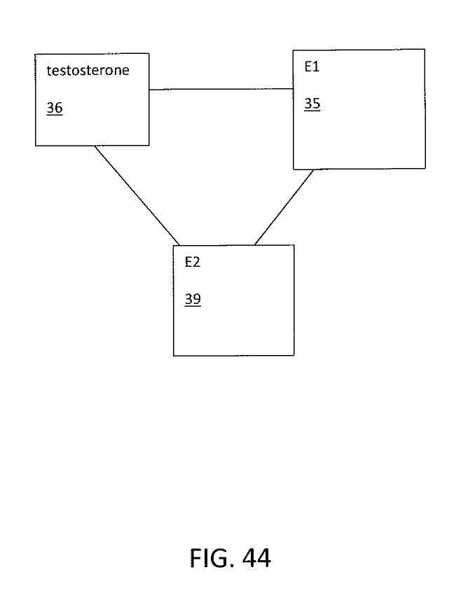

Another example of the determination of homeostatic levels of the sets of modulated pairs discussed above is now discussed. The pair of testosterone 36 and E1 35 is modulated by E2 39 and plays a role in the formation of Estrogen as it relates to males. This relationship is shown in FIG. 44.

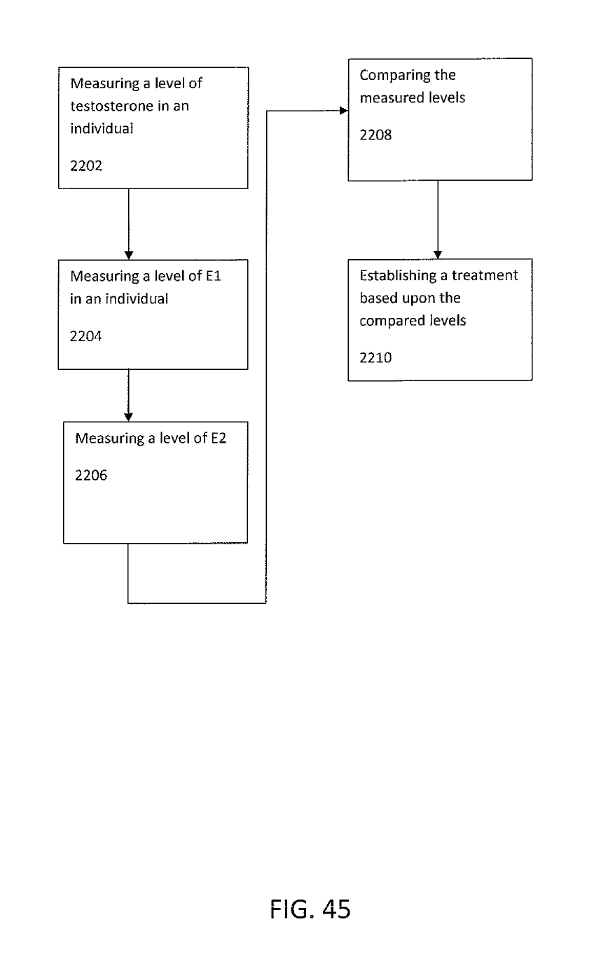

FIG. 45 shows the method of evaluating the homeostatic levels of the modulated pairs in order to provide a base index for administering medication and performing analysis. In step 2202, the level of testosterone 36 is measured in an individual. The level of testosterone 36 may be measured in the same manner as the substances discussed above.

The measured level of testosterone 36 in the body or environment may be expressed in various units depending upon the test employed in order to measure the level of the testosterone 36. This level can be expressed by the variable X. There is a preferred range in which X may fall that varies depending upon the type of test that is used in order to measure the level of testosterone 36. In an embodiment of the present invention a standard type of test is used.

In step 2204, the level of E1 35 in an individual is measured. This level can be measured using one of the various processes listed above. The measured level of E1 35 is preferably expressed in the same units of measurement as that used in the measurement of testosterone 36 so as to more readily compare the levels with respect to each other in order to obtain a base level. This level can be expressed by the variable Y. There is a preferred range in which Y may fall that varies depending upon the type of test that is used in order to measure the level of E1 35. In an embodiment of the present invention a standard type of test is used, such as a blood test.

In step 2206, the level of E2 39 in an individual is measured. This level can be measured using one of the various processes listed above. The measured level of E2 39 is preferably expressed in the same units of measurement as that used in the measurement of testosterone 36 so as to more readily compare the levels with respect to each other in order to obtain a base level. This level can be expressed by the variable Z. There is a preferred range in which Z may fall that varies depending upon the type of test that is used in order to measure the level of Z. In an embodiment of the present invention a standard type of test is used.

In step 2208, the measured levels X, Y and Z are then compared to standard levels. This may be done in a number of different ways. One possible way in which this can be accomplished is to total the values of all of the levels (X+Y+Z) in order to establish sum level C. This sum level C can be used in step 2210 in order to establish a diagnosis of the individual that can be used in determining the provision of medication and other matters related to the measured levels. In particular the levels may be used to determine if more E2 39 may need to be supplied in order to place the level of testosterone 36 and E1 35 back to proper levels and to further promote neuron synthesis. A value for C that falls below or above the predetermined level may signal the need to administer additional E2 39 to the individual. The E2 39 may be administered via pill form or some other acceptable means. Alternatively, it may be suggested that an individual's behavior be modified in order to reduce levels of one of the substances.

The assessment of the modulated substances enables the provision of consulting/counseling services relative to the biological foundation for and between the substances associated in playing a role in the process of the formation of Estrogen in males.

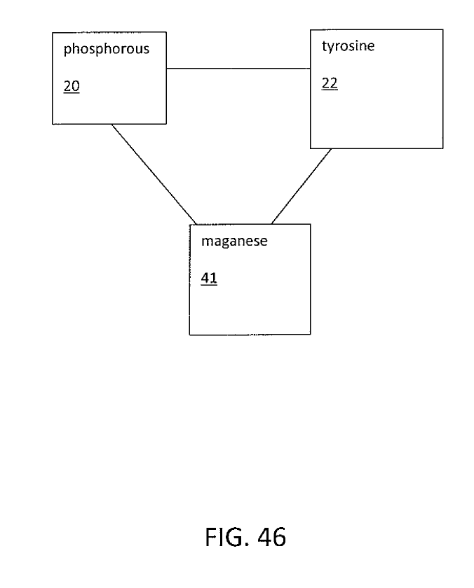

Another example of the determination of homeostatic levels of the sets of modulated pairs discussed above is now discussed. The pair of phosphorous 20 and tyrosine 22 is modulated by manganese 41 and plays a role in a signaling of the Abl protein. This relationship is shown in FIG. 46.

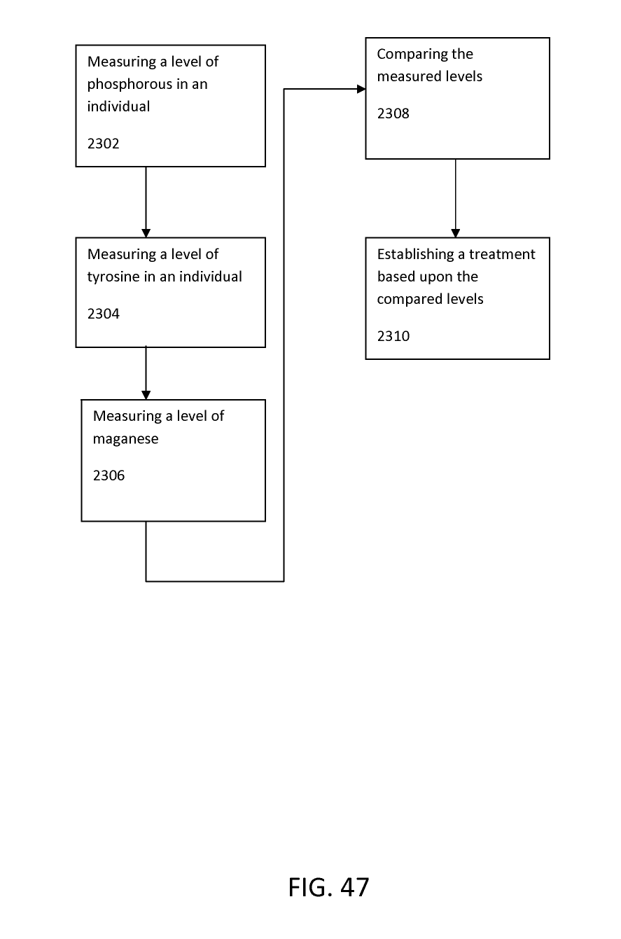

FIG. 47 shows the method of evaluating the homeostatic levels of the modulated pairs in order to provide a base index for administering medication and performing analysis. In step 2302, the level of phosphorous 20 is measured in an individual. The level of phosphorous 20 may be measured in the same manner as the substances discussed above.

The measured level of phosphorous 20 in the body or environment may be expressed in various units depending upon the test employed in order to measure the level of the phosphorous 20. This level can be expressed by the variable X. There is a preferred range in which X may fall that varies depending upon the type of test that is used in order to measure the level of phosphorous 20. In an embodiment of the present invention a standard type of test is used.

In step 2304, the level of tyrosine 22 in an individual is measured. This level can be measured using one of the various processes listed above. The measured level of tyrosine 22 is preferably expressed in the same units of measurement as that used in the measurement of phosphorous 20 so as to more readily compare the levels with respect to each other in order to obtain a base level. This level can be expressed by the variable Y. There is a preferred range in which Y may fall that varies depending upon the type of test that is used in order to measure the level of tyrosine 22. In an embodiment of the present invention a standard type of test is used, such as a blood test.

In step 2306, the level of manganese 41 in an individual is measured. This level can be measured using one of the various processes listed above. The measured level of manganese 41 is preferably expressed in the same units of measurement as that used in the measurement of phosphorous 20 so as to more readily compare the levels with respect to each other in order to obtain a base level. This level can be expressed by the variable Z. There is a preferred range in which Z may fall that varies depending upon the type of test that is used in order to measure the level of Z. In an embodiment of the present invention a standard type of test is used.

In step 2308, the measured levels X, Y and Z are then compared to standard levels. This may be done in a number of different ways. One possible way in which this can be accomplished is to total the values of all of the levels (X+Y+Z) in order to establish sum level C. This sum level C can be used in step 2310 in order to establish a diagnosis of the individual that can be used in determining the provision of medication and other matters related to the measured levels. In particular the levels may be used to determine if more manganese 41 may need to be supplied in order to place the level of phosphorous 20 and tyrosine 22 back to proper levels and to further promote neuron synthesis. A value for C that falls below or above the predetermined level may signal the need to administer additional manganese 41 to the individual. The manganese 41 may be administered via pill form or some other acceptable means. Alternatively, it may be suggested that an individual's behavior be modified in order to reduce levels of one of the substances.

The assessment of the modulated substances enables the provision of consulting/counseling services relative to the biological foundation for and between the substances associated in playing a role in the process of a signaling of the Abl protein.

It is to be understood, however, that even though numerous characteristics and advantages of the present invention have been set forth in the foregoing description, together with details of the structure and function of the invention, the disclosure is illustrative only, and changes may be made in detail, especially in matters of shape, size and arrangement of parts within the principles of the invention to the full extent indicated by the broad general meaning of the terms in which the appended claims are expressed.

What is claimed is:

1. A method for establishing a treatment for promoting the formation of Vitamin C in an individual:
measuring a level of calcium in the individual;
measuring a level of cysteine in the individual;
measuring a level of magnesium in the individual;
comparing the measured levels; and
establishing a treatment based upon the measured levels.

2. The method of claim 1, wherein the treatment comprises administering magnesium to the individual.

3. The method of claim 1, wherein the treatment comprises reducing intake of magnesium.

4. The method of claim 1, wherein the measuring is accomplished with a blood test.

5. The method of claim, wherein the measuring is accomplished with a mouth swab.

6. The method of claim 1, wherein test data is stored in a central computer and compiled.

7. The method of claim 1, wherein the levels of calcium, cysteine and magnesium are re-measured.

8. A method for establishing a treatment for promoting the formation of Vitamin D in an individual:
measuring a level of phosphorous in the individual;
measuring a level of tyrosine in the individual;
measuring a level of calcium in the individual;
comparing the measured levels; and
establishing a treatment based upon the measured levels.

9. The method of claim 8, wherein the treatment comprises administering calcium to the individual.

10. The method of claim 8, wherein the treatment comprises reducing intake of calcium.

11. The method of claim 8, wherein the measuring is accomplished with a blood test.

12. The method of claim 8, wherein the measuring is accomplished with a mouth swab.

13. The method of claim 8, wherein test data is stored in a central computer and compiled.

14. The method of claim 8, wherein the levels of phosphorous, tyrosine and calcium are re-measured.

15. A method for establishing a treatment for promoting the formation of Vitamin B9 in an individual:
measuring a level of phosphorus in the individual;
measuring a level of cysteine in the individual;
measuring a level of sulfur in the individual;
comparing the measured levels; and
establishing a treatment based upon the measured levels.

16. The method of claim 15, wherein the treatment comprises administering sulfur to the individual.

17. The method of claim 15, wherein the treatment comprises reducing intake of sulfur.

18. The method of claim 15, wherein the measuring is accomplished with a blood test.

19. The method of claim 15, wherein the measuring is accomplished with a mouth swab.

20. The method of claim 15, wherein test data is stored in a central computer and compiled.
